(19) United States
(12) Patent Application Publication
(10) Pub. No.: US 2022/0194951 A1
(43) Pub. Date: Jun. 23, 2022

(54) DIHYDROPYRIMIDINE COMPOUND AND APPLICATION THEREOF AS DRUG

(71) Applicant: SUNSHINE LAKE PHARMA CO., LTD., Dongguan, Guangdong (CN)

(72) Inventors: Qingyun REN, Dongguan (CN); Yingjun ZHANG, Dongguan (CN); Xinchang LIU, Dongguan (CN); Siegfried GOLDMANN, Wuppertal (DE); Guanghua YAN, Dongguan (CN); Meng WANG, Dongguan (CN); Douxing LEI, Dongguan (CN); Feng LI, Dongguan (CN)

(73) Assignee: SUNSHINE LAKE PHARMA CO., LTD., Dongguan, Guangdong (CN)

(21) Appl. No.: 17/605,456

(22) PCT Filed: Apr. 29, 2020
(86) PCT No.: PCT/CN2020/087700

§ 371 Date: Oct. 21, 2021

(30) Foreign Application Priority Data
Apr. 30, 2019 (CN) 201910360129.6

(51) Int. Cl. C07D 487/04; A61K 45/06; A61K 31/506; A61P 31/20

(52) U.S. Cl. CPC C07D 487/04; A61P 31/20; A61K 31/506; A61K 45/06

(57) ABSTRACT

A dihydropyrimidine compound and application thereof as a drug, in particular, application as a drug for treating and preventing hepatitis B. Specifically, a compound represented by general formula (I) or (Ia) or a stereoisomer, a tautomer, an oxynitride, a solvate, a metabolite, a pharmaceutically acceptable salt or a prodrug thereof, wherein variables are as defined in the description. Use of the compound represented by general formula (I) or (Ia) or the stereoisomer, the tautomer, the oxynitride, the solvate, the metabolite, or the pharmaceutically acceptable salt thereof as a drug, in particular use as a drug for treating and preventing hepatitis B.

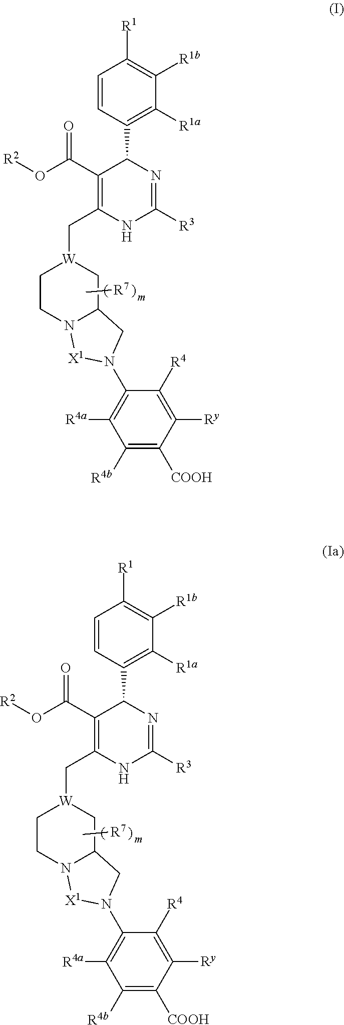

### CROSS-REFERENCE TO RELATED APPLICATION

This is a U.S. national stage application of the International Patent Application No. PCT/CN2020/087700, filed Apr. 29, 2020, which claims priority to Chinese Patent Application No. 201910360129.6, filed Apr. 30, 2019, both of which are incorporated herein by reference in their entirety.

### FIELD OF THE INVENTION

The invention belongs to medicine field. Specifically, the present invention relates to a dihydropyrimidine compound and uses thereof in medicine, especially as a medicament for treating and/or preventing hepatitis B. The invention also relates to compositions of dihydropyrimidine compounds together with other antiviral agents, and applications in treating and preventing hepatitis B virus (HBV) infection diseases.

### BACKGROUND OF THE INVENTION

The hepatitis B virus belongs to the family of hepadnaviridae. It can cause acute and/or progressive chronic diseases. Many other clinical manifestations in the pathological morphology can be also caused by HBV—in particularchronic hepatitis, cirrhosis and hepatocellular carcinoma. Additionally, coinfection with hepatitis D virus may have adverse effects on the progress of the disease.

The conventional medicaments approved to be used for treating chronic hepatitis are interferon and lamivudine. However, the interferon has just moderate activity but has an adverse side reaction. Although lamivudine has good activity, its drug resistance develops rapidly during the treatment and relapse effects often appear after the treatment has stopped. The IC₅₀ value of lamivudine (3-TC) is 300 nM (Science, 299(2003), 893-896).

Deres et al. have reported heteroaryl-substituted dihydropyrimidine (HAP) compounds represented by Bay41-4109 and Bay39-5493, these compounds can inhibit the replication of HBV by preventing the formation of normal nucleocapsid. Bay41-4109 has a good drug metabolism properties in clinical research (Science, 299(2003), 893-896). The study of these compounds' mechanism indicates that through reacting with 113-143 amino acid residues of a core protein, heteroaryl-substituted dihydropyrimidine compounds have changed the angle between dimers which can form nucleocapsids, resulting in the formation of unstably expanded nucleocapsids, which accelerate the degradation of the core protein (Biochem. Pharmacol, 2003, 66, 2273-2279).

Novel compounds with effective antiviral effects are still desired at present, especially drugs used for the treatment and/or prevention of hepatitis B.

### SUMMARY OF THE INVENTION

The present invention relates to a novel dihydropyrimidine compound and uses thereof in the preparation of a medicament for the treatment and prevention of HBV infection. In particular, the present invention relates to a novel dihydropyrimidine compound and a pharmaceutically acceptable composition thereof, the compound has advantages of good solubility, good stability, basically no induction effect on liver drug enzymes, low toxicity, etc., especially has very good pharmacokinetic properties. The compound of the present invention can effectively inhibit HBV infection, and has a good application prospect in anti-HBV.

In one aspect, provided herein is a compound having Formula (I) or (Ia), or a stereoisomer, a tautomer, an N-oxide, a solvate, a metabolite, a pharmaceutically acceptable salt or a prodrug thereof,

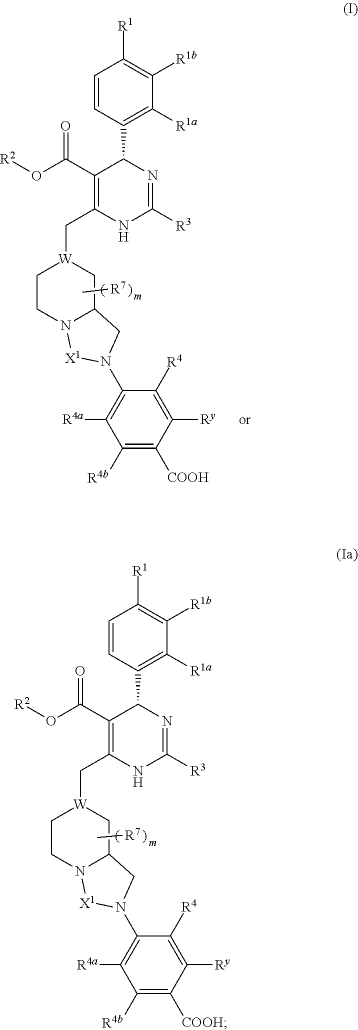

wherein, each of R¹, R^{1b} and R^{1a} is independently hydrogen, deuterium, F, Cl, Br, I, cyano, methyl, ethyl, methoxy, ethoxy, methylamino, ethylamino, nitro, 4-trifluoromethylphenyl, 3,5-bis(trifluoromethyl)phenyl or trifluoromethyl;

R² is C₁₋₆ alkyl or C₁₋₆ haloalkyl;

R³ is phenyl, imidazolyl, furyl, thienyl or thiazolyl, wherein the phenyl, imidazolyl, furyl, thienyl and thiazolyl are each independently unsubstituted or substituted with 1, 2, 3, 4 or 5 substituents selected from the following: deuterium, F, Cl, Br, OH, CN, C₁₋₆ alkyl, hydroxy C₁₋₆ alkyl, C₁₋₆ alkyl-OC(═O)—, C₁₋₆ alkyl-OC(═O)—C₁₋₆ alkylene, HOOC—C₁₋₆ alkylene, C₁₋₆ alkoxy-C₁₋₆ alkylene and C₁₋₆ alkyl-S(═O)2-;

W is CH or N;

X¹ is —C(═O)—, —S(═O)₂— or —(CR⁵R⁶) each of R^{4a}, R^{4b}, R⁵ and R⁶ is independently hydrogen, deuterium, F, Cl, Br, amino, C₁₋₆ alkyl, NH₂C(═O)—, C₁₋₆ alkyl —OC(═O)—, hydroxyC₁₋₆ alkyl, C₁₋₄ alkoxy C₁₋₄ alkylene or C₁₋₆ haloalkyl;

each R⁷ is independently hydrogen, deuterium, F, Cl, Br, amino, C₁₋₆ alkyl, NH₂C(═O)—, C₁₋₆ alkyl —OC(═O)—, carboxy, carboxy C₁₋₆ alkylene, hydroxy C₁₋₆ alkyl, C₁₋₄ alkoxy C₁₋₄ alkylene or C₁₋₆ haloalkyl;

R^{y} is hydrogen, R⁴ is methyl, ethyl, n-propyl, methoxy, ethoxy, n-propoxy, isopropoxy, F or Cl; or

R^{y} is F or Cl, R⁴ is hydrogen, F or Cl;

m is 0, 1, 2, 3 or 4;

j is 1, 2, or 3.

In some embodiments, the R² is methyl, ethyl, n-propyl, isopropyl, monofluoromethyl, difluoromethyl or trifluoromethyl;

R³ is phenyl, imidazolyl, furyl, thienyl or thiazolyl, wherein the phenyl and thiazolyl are each independently unsubstituted or substituted with 1, 2, 3, 4 or 5 substituents selected from the following: deuterium, F, Cl, Br, OH, CN, methyl, ethyl, n-propyl, isopropyl, tert-butyl, hydroxy C₁₋₄ alkyl, C₁₋₄ alkyl —OC(═O)—, C₁₋₄ alkyl —OC(═O)—C₁₋₃ alkylene, HOOC—C₁₋₃ alkylene, C₁₋₄ alkoxy-C₁₋₃ alkylene and C₁₋₄ alkyl —S(═O)2-.

In some embodiments, each of R^{4a}, R^{4b}, R⁵ and R⁶ is independently hydrogen, deuterium, F, Cl, Br, amino, methyl, ethyl, n-propyl, isopropyl, n-butyl, tert-butyl, NH₂C(═O)—, C₁₋₄ alkyl —OC(═O)—, hydroxy C₁₋₄ alkyl, C₁₋₄ alkoxy C₁₋₂ alkylene or C₁₋₄ haloalkyl;

Each R⁷ is independently deuterium, F, Cl, Br, amino, methyl, ethyl, n-propyl, isopropyl, n-butyl, tert-butyl, NH₂C(═O)—, C₁₋₄ alkyl —OC(═O)—, carboxy, carboxy C₁₋₄ alkylene, hydroxy C₁₋₄ alkyl, C₁₋₄ alkoxy C₁₋₂ alkylene or C₁₋₄ haloalkyl.

In another aspect, provided herein is a pharmaceutical composition comprising the compound of the invention, and a pharmaceutically acceptable adjuvant.

In some embodiments, the pharmaceutical composition disclosed herein further comprises other anti-HBV drug.

In some embodiments of the pharmaceutical composition disclosed herein, wherein the other anti-HBV drug is a HBV polymerase inhibitor, an immunomodulator or an interferon.

In some embodiments of the pharmaceutical composition disclosed herein, wherein the other anti-HBV drug is lamivudine, telbivudine, tenofovir, entecavir, adefovir dipivoxil, alfaferone, alloferon, celmoleukin, clevudine, emtricitabine, famciclovir, feron, hepatect CP, intefen, interferon α-1b, interferon α, interferon α-2a, interferon β-1a, interferon α-2, interleukin-2, mivotilate, nitazoxanide, peginterferona-2a, ribavirin, roferon-A, sizofiran, Euforavac, rintatolimod, Phosphazid, Heplisav, interferon α-2b, levamisole, or propagermanium.

In another aspect, also provided herein is use of the compound or the pharmaceutical composition disclosed herein in the manufacture of a medicament for preventing, treating or lessening a virus disease in a patient.

In some embodiments of the use, wherein the virus disease disclosed herein is hepatitis B infection or a disease caused by hepatitis B infection.

In other embodiments of the use, wherein the disease caused by hepatitis B infection disclosed herein is hepatic cirrhosis or hepatocellular carcinogenesis.

In other aspect, provided herein is use of the compound or the pharmaceutical composition in the manufacture of a medicament for preventing, treating or lessening a HBV disease in a patient, comprising administering to the patient a therapeutically effective amount of the compound or the pharmaceutical composition of the invention.

In other aspect, the present invention relates to a method of preventing, treating or lessening a HBV disease in a patient, comprising administering a pharmaceutically acceptable effective amount of the compound to a patient.

In other aspect, the present invention relates to a method of preventing, treating or lessening a HBV disease in a patient, comprising administering a pharmaceutically acceptable effective amount of the pharmaceutical composition containing the compound of the invention to a patient.

In other aspect, provided herein is use of the compound disclosed herein in the manufacture of a medicament for preventing, managing or treating an HBV disease in a patient, and lessening the severity thereof.

In other aspect, provided herein is use of the composition containing the compound disclosed herein in the manufacture of a medicament for preventing, managing or treating a HBV disease in a patient, and lessening the severity thereof.

In other aspect, provided herein is a method of inhibiting HBV infection, comprising contacting cells with the compound or pharmaceutical composition of the invention in a dose effective to inhibit HBV. In other embodiments, the method further comprises contacting the cell with another anti-HBV therapeutic agent.

In other aspect, the present invention relates to a method of treating a HBV disease in a patient, comprising administrating a therapeutically effective amount of the compound or composition thereof to a patient in need. In other embodiments, the method further comprises administering an effective therapeutic dose of other anti-HBV drug to a patient in need.

In other aspect, the present invention relates to a method of inhibiting a HBV infection in a patient, comprising administrating a therapeutically effective amount of the compound or composition thereof to a patient in need. In other embodiments, the method further comprises administering an effective therapeutic dose of other anti-HBV drug to a patient in need.

In other aspect, provided herein is a method of preparing, separating and purifying the compounds contained in Formula (I) or (Ia).

The foregoing merely summarizes certain aspects disclosed herein and is not intended to be limiting in nature. These aspects and other aspects and embodiments are described more fully below.

### DETAILED DESCRIPTION OF THE INVENTION

### Definitions and General Terminology

The invention will list the documents corresponding to the determined specific content in detail, and the examples are accompanied by illustrations of structural formulas and chemical formulas. The invention prospectively covers all options, variants and equivalents, which may be included in the current invention field as defined by the claims. Those skilled in the art will recognize many methods and substances similar or equivalent to those described herein, which can be applied in the practice of the present invention. The invention is by no means limited to the description of methods and substances. There are many documents and similar materials that are different or inconsistent with the application of the present invention, including but not limited to the definition of terms, the usage of terms, the described technology, or the scope controlled by the application of the present invention.

The invention will apply the following definitions unless otherwise indicated. For purposes of this invention, the chemical elements are identified in accordance with the Periodic Table of the Elements, CAS version, and the Handbook of Chemistry and Physics, 75th Ed. 1994. Additionally, general principles of organic chemistry are described in “Organic Chemistry”, Thomas Sorrell, University Science Books, Sausalito: 1999, and “March's Advanced Organic Chemistry,” by Michael B. Smith and Jerry March, John Wiley&Sons, New York: 2007, the entire contents of which are hereby incorporated by reference.

As described herein, compounds disclosed herein may optionally be substituted with one or more substituents, such as are illustrated generally above, or as exemplified by particular classes, subclasses, and species of the invention.

In general, the term “substituted” refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position.

At each part of the present specification, substitutes of compounds herein are disclosed in groups or in ranges. It is specifically intended that the invention includes each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

The term “alkyl” or “alkyl group” refers to a saturated linear or branched-chain monovalent hydrocarbon radical of 1 to 20 carbon atoms, wherein the alkyl radical may be optionally and independently substituted with one or more substituents described herein. In some embodiments, the alkyl group contains 1-12 carbon atoms. In other embodiments, the alkyl group contains 1-10 carbon atoms. In other embodiments, the alkyl group contains 1-8 carbon atoms. In still other embodiments, the alkyl group contains 1-6 carbon atoms. In yet other embodiments, the alkyl group contains 1-4 carbon atoms and in still yet other embodiments, the alkyl group contains 1-3 carbon atoms. Some further non-limiting examples of the alkyl group include, methyl (Me, —CH₃), ethyl (Et, —CH₂CH₃), n-propyl (n-Pr, —CH₂CH₂CH₃), isopropyl (i-Pr, —CH(CH₃)₂), n-butyl (n-Bu, —CH₂CH₂CH₂CH₃), 2-methylpropyl or isobutyl (i-Bu, —CH₂CH(CH₃)₂), 1-methylpropyl or sec-butyl (s-Bu, —CH(CH₃)CH₂CH₃), tert-butyl (t-Bu, —C(CH₃)₃), n-pentyl (—CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), n-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, n-heptyl and n-octyl, etc.

The term “alkylene” refers to a saturated divalent or multivalent hydrocarbon group derived from a straight or branched chain saturated hydrocarbon by the removal of two or more hydrogen atoms. Unless otherwise specified, the alkylene group contains 1-12 carbon atoms. In some embodiments, the alkylene group contains 1-6 carbon atoms. In other embodiments, the alkylene group contains 1-4 carbon atoms. In still other embodiments, the alkylene group contains 1-3 carbon atoms. In yet other embodiments, the alkylene group contains 1-2 carbon atoms. Such examples of the alkylene include but are not limited to methylene (—CH₂—), ethylene (—CH₂CH₂—), n-propylene (—CH₂CH₂CH₂—), isopropylene (—CH(CH₃)CH₂—), and the like.

The terms “hydroxyalkyl” and “hydroxyalkoxy” mean alkyl or alkoxy, as the case may be, substituted by one or more hydroxy groups, wherein “hydroxyalkyl” and “hydroxyalkylene” and “hydroxyalkyl” can be used interchangeably. Such examples include but are not limited to, hydroxymethyl (—CH₂OH), hydroxyethyl (—CH₂CH₂OH, —CHOHCH₃), and hydroxypropyl (e.g., —CH₂CH₂CH₂OH, —CH₂CHOHCH₃, —CHOHCH₂CH₃), hydroxymethoxy (—OCH₂OH), etc. The terms “haloalkyl”, “haloalkenyl” or “haloalkoxy” refer to alkyl, alkenyl or alkoxy substituted with one or more halogen atoms. Wherein the alkyl, alkenyl and alkoxy are as defined herein. Such examples include, but are not limited to, difluoroethyl (—CH₂CHF₂, —CF₂CH₃, —CHFCH₂F), trifluoroethyl (—CH₂CF₃, —CF₂CH₂F, —CFHCHF₂), trifluoromethyl (—CF₃), trifluoromethoxy (—OCF₃), fluorovinyl (—CH═CHF, —CF═CH₂), etc.

The term “alkoxy” refers to an alkyl group, as previously defined, attached to parent molecular moiety via an oxygen atom. Unless otherwise specified, the alkoxy group contains 1-12 carbon atoms. In some embodiments, the alkoxy group contains 1-8 carbon atoms. In other embodiments, the alkoxy group contains 1-6 carbon atoms. In still other embodiments, the alkoxy group contains 1-4 carbon atoms. In yet other embodiments, the alkoxy group contains 1-3 carbon atoms. The alkoxy group may be optionally substituted with one or more substituents disclosed herein.

Some non-limiting examples of the alkoxy group include, methoxy (MeO, —OCH₃), ethoxy (EtO, —OCH₂CH₃), 1-propoxy (n-PrO, n-propoxy, —OCH₂CH₂CH₃), 2-propoxy (i-PrO, i-propoxy, —OCH(CH₃)₂), 1-butoxy (n-BuO, n-butoxy, —OCH₂CH₂CH₂CH₃), 2-methyl-1-propoxy (i-BuO, i-butoxy, —OCH₂CH(CH₃)₂), 2-butoxy (s-BuO, s-butoxy, —OCH(CH₃)CH₂CH₃), 2-methyl-2-propoxy (t-BuO, t-butoxy, —OC(CH₃)₃), 1-pentoxy (n-pentoxy, —OCH₂CH₂CH₂CH₂CH₃), 2-pentoxy (—OCH(CH₃)CH₂CH₂CH₃), 3-pentoxy (—OCH(CH₂CH₃)₂), 2-methyl-2-butoxy (—OC(CH₃)₂CH₂CH₃), 3-methyl-2-butoxy (—OCH(CH₃)CH(CH₃)₂), 3-methyl-1-butoxy (—OCH₂CH₂CH(CH₃)₂), 2-methyl-1-butoxy (—OCH₂CH(CH₃)CH₂CH₃), etc.

The term “halogen” or “halogen atom” means F, Cl, Br or I.

The term “unsaturated” refers to a moiety having one or more units of unsaturation.

The term “aryl” refers to monocyclic, bicyclic and tricyclic carbocyclic ring systems having a total of 6 to 14 ring members, or 6 to 12 ring members, or 6 to 10 ring members, wherein at least one ring in the system is aromatic, wherein each ring in the system contains 3 to 7 ring members and that has a single point or multipoint of attachment to the rest of the molecule. The term “aryl” and “aromatic ring” can be used interchangeably herein. Examples of the aryl group may include phenyl, naphthyl and anthracenyl. The aryl group may be independently and optionally substituted by one or more substituents disclosed herein.

The term “heteroaryl” refers to monocyclic, bicyclic and tricyclic carbocyclic ring systems having a total of 5 to 12 ring members, wherein at least one ring in the system is aromatic ring, and in which at least one aromatic ring contains one or more heteroatoms, and wherein each ring in the system contains 5 to 7 ring members and that has a single point or multipoint of attachment to the rest of the molecule. The term “heteroaryl” and “aromatic heterocyclic”, “heteroaryl ring” or “heteroaromatic compound” can be used interchangeably herein. In some embodiments, the heteroaryl is a 5-7 membered monocyclic heteroaryl comprising 1, 2, 3 or 4 heteroatoms independently selected from nitrogen, sulfur, and oxygen. In some embodiments, the heteroaryl is a 5-6 membered monocyclic heteroaryl comprising 1, 2, 3 or 4 heteroatoms independently selected from nitrogen, sulfur, and oxygen. In some embodiments, the heteroaryl is a 7-12 membered bicyclic heteroaryl comprising 1, 2, 3 or 4 heteroatoms independently selected from nitrogen, sulfur, and oxygen. In some embodiments, the heteroaryl is a 8-10 membered bicyclic heteroaryl comprising 1, 2, 3 or 4 heteroatoms independently selected from nitrogen, sulfur, and oxygen. In some embodiments, the heteroaryl is a 9-10 membered bicyclic heteroaryl comprising 1, 2, 3 or 4 heteroatoms independently selected from nitrogen, sulfur, and oxygen.

Some non-limiting examples of heteroaryl include the following monocyclic groups: 1,2,4-oxadiazole-5(4H)-thione, 1,2,4-thiadiazole-5(4H)-keto, 1,2,4-oxadiazole-5(4H)-keto, 1,3,4-oxadiazole-2(3H)-thione, 1H-1,2,4-triazole-5(4H)-keto, 2-furanyl, 3-furanyl, N-imidazolyl, 2-imidazoly, 4-imidazoly, 5-imidazoly, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g. 3-pyridazinyl), 2-thiazolyl, 4-thiazolyl, 5-thiazolyl, tetrazolyl (e.g. 5-tetrazolyl), triazolyl (e.g. 2-triazolyl and 5-triazolyl), 2-thienyl, 3-thienyl, pyranyl, pyrazolyl (e.g. 2-pyrazolyl), isothiazolyl, 1,2,3-oxadiazolyl, 1,2,5-oxadiazolyl, 1,2,4-oxadiazolyl, 1,2,3-triazolyl, 1,2,3-thiodiazolyl, 1,3,4-thiodiazolyl, 1,2,5-thiodiazolyl, pyrazinyl, 1,3,5-triazinyl, diazolyl, thiadiazolyl, triazinyl, etc.; and the following bicyclic groups: benzothiazolyl, benzimidazolyl, benzofuranyl, benzothienyl, indolyl (e.g. 2-indolyl), purinyl, quinolinyl (e.g. 2-quinolinyl, 3-quinolinyle, 4-quinolinyl), isoquinolinyl (e.g. 1-isoquinolinyl, 3-isoquinolinyl or 4-isoquinolinyl), etc.

The term “M-M₁ membered” means that the cyclic group consisting of M-M₁ ring atoms, and the ring atoms include carbon atoms and/or O, N, S, P and other heteroatoms. For example, “3-6 membered heterocyclyl” refers to a heterocyclic group consisting of 3, 4, 5, or 6 atoms.

The terms “alkoxyalkyl” and “alkoxyalkylene” which can be used interchangeably refer that an alkyl group can be substituted with one or more alkoxy groups which may be the same or different, wherein the alkoxy and alkane groups are as defined herein. Some non-limiting examples of such group include cyclohexylmethyl, cyclopropylethyl, methoxyethyl, ethoxymethyl, etc.

As described herein, a bond drawn from a substituent to the center of one ring within a ring system (as shown in Formula a) represents that the substituent can be substituted at any substitutable position on the ring, as shown in Formula b, c, d, e, f, g and h;

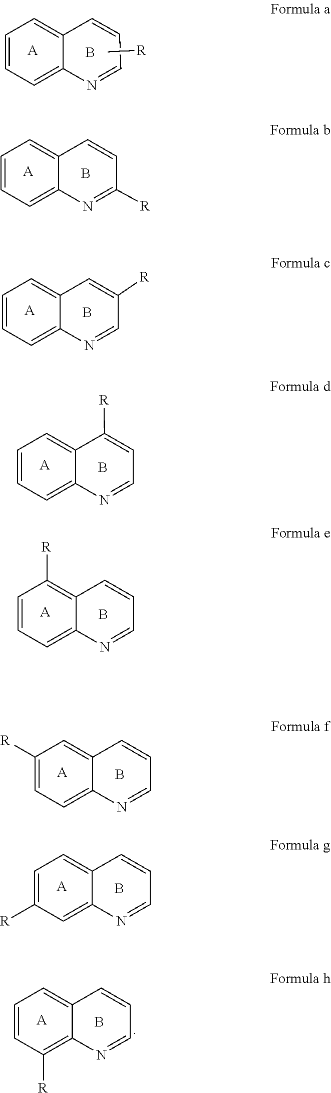

Furthermore, unless otherwise stated, the phrase “each . . . is independently” is used interchangeably with the phrase “each (of) . . . and . . . is independently”. It should be understood broadly that the specific options expressed by the same symbol are independent of each other in different radicals; or the specific options expressed by the same symbol are independent of each other in same radicals. For example, as shown in Formula p, multiple R⁷ are independent of each other,

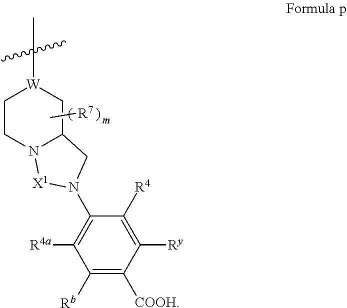

Unless otherwise stated, the structures depicted herein are also meant to include all isomeric (e.g., enantiomeric, diastereomeric, and geometric or conformational) forms of the structure; for example, the R and S configurations for each asymmetric center, (Z) and (E) double bond isomers, and (Z) and (E) conformational isomers. Therefore, single stereochemical isomers as well as enantiomeric, diastereomeric, or geometric (or conformational) mixtures of the present compounds are within the scope disclosed herein.

The term “prodrug” refers to a compound that is transformed in vivo into a compound of Formula (I). Such a transformation can be affected, for example, by hydrolysis of the prodrug form in blood or enzymatic transformation to the parent form in blood or tissue. Prodrugs of the compounds disclosed herein may be, for example, esters. Some common esters which have been utilized as prodrugs are phenyl esters, aliphatic (C₁₋₂₄) esters, acyloxymethyl esters, carbonates, carbamates and amino acid esters. For example, a compound disclosed herein that contains a hydroxy group may be acylated at this position in its prodrug form. Other prodrug forms include phosphates, such as, those phosphate compounds derived from the phosphonation of a hydroxy group on the parent compound. A thorough discussion of prodrugs is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, J. Rautio et al., Prodrugs: Design and Clinical Applications, Nature Review Drug Discovery, 2008, 7, 255-270, and S. J. Hecker et al., Prodrugs of Phosphates and Phosphonates, Journal of Medicinal Chemistry, 2008, 51, 2328-2345, all of which are incorporated herein by reference in their entireties.

Unless otherwise stated, all tautomeric forms of the compounds disclosed herein are within the scope of the invention. Additionally, unless otherwise stated, structures depicted herein are also meant to include compounds that differ only in the presence of one or more isotopically enriched atoms.

A “metabolite” is a product produced through metabolism in the body of a specified compound or salt thereof. The metabolites of a compound may be identified using routine techniques known in the art and their activities may be determined using tests such as those described herein. Such products may result for example from oxidation, reduction, hydrolysis, amidation, deamidation, esterification, deesterification, enzyme cleavage, and the like, of the administered compound. Accordingly, the invention includes metabolites of compounds disclosed herein, including metabolites produced by contacting a compound disclosed herein with a mammal for a sufficient time period.

Stereochemical definitions and conventions used herein generally follow S. P. Parker Ed., McGraw-Hill Dictionary of Chemical Terms (1984) McGraw-Hill Book Company, New York and Eliel et al., “Stereochemistry of Organic Compounds”, John Wiley & Sons, Inc., New York, 1994. The compounds disclosed herein may contain asymmetric or chiral centers, and therefore exist in different stereoisomeric forms. It is intended that all stereoisomeric forms of the compounds disclosed herein, including, but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The term “racemic mixture” or “racemate” refers to an equimolar mixture of two enantiomeric species, devoid of optical activity.

The term “tautomer” or “tautomeric form” refers to structural isomers of different energies which are interconvertible via a low energy barrier. Some non-limiting examples of proton tautomers (also known as prototropic tautomers) include interconversions via migration of a proton, such as keto-enol and imine-enamine isomerizations. Valence tautomers include interconversions by reorganization of some of the bonding electrons. Unless otherwise stated, all tautomeric forms of the compounds disclosed herein are within the scope of the invention.

A “pharmaceutically acceptable salts” refers to organic or inorganic salts of a compound disclosed herein. Pharmaceutically acceptable salts are well known in the art. For example, Berge et al., describe pharmaceutically acceptable salts in detail in J. Pharmacol Sci, 1977, 66:1-19, which is incorporated herein by reference. Some non-limiting examples of pharmaceutically acceptable and nontoxic salts include salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid and malonic acid or by using other methods used in the art such as ion exchange. Other pharmaceutically acceptable salts include adipate, malate, 2-hydroxy propionate, alginate, ascorbate, aspartate, benzenesulfonate, benzoate, bisulfate, borate, butyrate, camphorate, camphorsulfonate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, laurylsulfate, malate, malonate, methanesulfonate, 2-naphthalenesulfonate, nicotinate, nitrate, oleate, palmitate, pamoate, pectinate, persulfate, 3-phenylpropionate, picrate, pivalate, propionate, stearate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. This invention also envisions the quaternization of any basic nitrogen-containing groups of the compounds disclosed herein. Water or oil soluble or dispersable products may be obtained by such quaternization. Representative alkali or alkaline earth metal salts include sodium, lithium, potassium, calcium, magnesium, and the like. Further pharmaceutically acceptable salts include appropriate and nontoxic ammonium, quaternary ammonium, and amine cations formed using counterions, such as halide, hydroxide, carboxylate, sulfate, phosphate, nitrate, C₁₋₈ sulfonate or aryl sulfonate.

The term “solvate” refers to an association or complex of one or more solvent molecules and a compound disclosed herein. Some non-limiting examples of the solvent that form solvates include water, isopropanol, ethanol, methanol, dimethylsulfoxide (DMSO), ethyl acetate, acetic acid and ethanolamine. The term “hydrate” refers to the complex where the solvent molecule is water.

The term “protecting group” or “Pg” refers to a substituent that is commonly employed to block or protect a particular functionality while reacting with other functional groups on the compound. For example, an “amino-protecting group” is a substituent attached to an amino group that blocks or protects the amino functionality in the compound. Suitable amino-protecting groups include acetyl, trifluoroacetyl, t-butoxy-carbonyl (BOC), benzyloxycarbonyl (CBZ) and 9-fluorene methyleneoxy-carbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include —CH₂CH₂SO₂Ph, cyanoethyl, 2-(trimethylsilyl)ethyl, 2-(trimethylsilyl) ethoxy-methyl, 2-(p-toluenesulfonyl) ethyl, 2-(p-nitrophenylsulfonyl)-ethyl, 2-(diphenylphosphino)-ethyl, nitroethyl and the like. For a general description of protecting groups and their use, see T. W. Greene, Protective Groups in Organic Synthesis, John Wiley & Sons, New York, 1991; and P. J. Kocienski, Protecting Groups, Thieme, Stuttgart, 2005.

### Description of Compounds of the Invention

The compounds of the present invention, and pharmaceutically acceptable compositions thereof, are effective in inhibiting HBV infection.

In one aspect, the present invention provides a compound having Formula (I) or (Ia), or a stereoisomer, a tautomer, an N-oxide, a solvate, a metabolite, a pharmaceutically acceptable salt or a prodrug thereof,

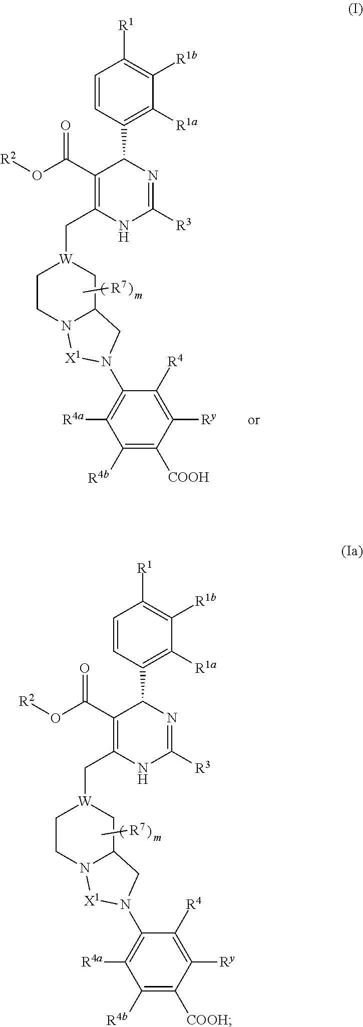

wherein, each of R¹, R^{1b} and R^{1a} is independently hydrogen, deuterium, F, Cl, Br, I, cyano, methyl, ethyl, methoxy, ethoxy, methylamino, ethylamino, nitro, 4-trifluoromethylphenyl, 3,5-bis(trifluoromethyl)phenyl or trifluoromethyl;

R² is C₁₋₆ alkyl or C₁₋₆ haloalkyl;

R³ is phenyl, imidazolyl, furyl, thienyl or thiazolyl, wherein the phenyl, imidazolyl, furyl, thienyl and thiazolyl are each independently unsubstituted or substituted with 1, 2, 3, 4 or 5 substituents selected from the following: deuterium, F, Cl, Br, OH, CN, C₁₋₆ alkyl, hydroxy C₁₋₆ alkyl, C₁₋₆ alkyl-OC(═O)—, C₁₋₆ alkyl-OC(═O)—C₁₋₆ alkylene, HOOC—C₁₋₆ alkylene, C₁₋₆ alkoxy-C₁₋₆ alkylene and C₁₋₆ alkyl-S(═O)₂—;

W is CH or N;

X¹ is —C(═O)—, —S(═O)₂— or —(CR⁵R⁶)ⱼ—;

each of R^{4a}, R^{4b}, R⁵ and R⁶ is independently hydrogen, deuterium, F, Cl, Br, amino, C₁₋₆ alkyl, NH₂C(═O)—, C₁₋₆ alkyl-OC(═O)—, hydroxyl C₁₋₆ alkyl, C₁₋₄ alkoxy, C₁₋₄ alkylene or C₁₋₆ haloalkyl;

each R⁷ is independently hydrogen, deuterium, F, Cl, Br, amino, C₁₋₆ alkyl, NH₂C(═O)—, C₁₋₆ alkyl —OC(═O)—, carboxy, carboxy C₁₋₆ alkylene, hydroxy C₁₋₆ alkyl, C₁₋₄ alkoxy C₁₋₄ alkylene or C₁₋₆ haloalkyl;

R^{y} is hydrogen, R⁴ is methyl, ethyl, n-propyl, methoxy, ethoxy, n-propoxy, isopropoxy, F or Cl; or

R^{y} is F or Cl, R⁴ is hydrogen, F or Cl;

m is 0, 1, 2, 3 or 4;

j is 1, 2, or 3.

In some embodiments, the R² is methyl, ethyl, n-propyl, isopropyl, monofluoromethyl, difluoromethyl or trifluoromethyl;

R³ is phenyl, imidazolyl, furyl, thienyl or thiazolyl, wherein the phenyl and thiazolyl are each independently unsubstituted or substituted with 1, 2, 3, 4 or 5 substituents selected from the following: deuterium, F, Cl, Br, OH, CN, methyl, ethyl, n-propyl, isopropyl, tert-butyl, hydroxy C₁₋₄ alkyl, C₁₋₄ alkyl —OC(═O)—, C₁₋₄ alkyl —OC(═O)—C₁₋₃ alkylene, HOOC—C₁₋₃ alkylene, C₁₋₄ alkoxy-C₁₋₃ alkylene and C₁₋₄ alkyl —S(═O)₂—.

In some embodiments, each of R^{4a}, R^{4b}, R⁵ and R⁶ is independently hydrogen, deuterium, F, Cl, Br, amino, methyl, ethyl, n-propyl, isopropyl, n-butyl, tert-butyl, NH₂C(═O)—, C₁₋₄ alkyl —OC(═O)—, hydroxy C₁₋₄ alkyl, C₁₋₄ alkoxy C₁₋₂ alkylene or C₁₋₄ haloalkyl;

each R⁷ is independently deuterium, F, Cl, Br, amino, methyl, ethyl, n-propyl, isopropyl, n-butyl, tert-butyl, NH₂C(═O)—, C₁₋₄ alkyl —OC(═O)—, carboxy, carboxy C₁₋₄ alkylene, hydroxy C₁₋₄ alkyl, C₁₋₄ alkoxy C₁₋₂ alkylene or C₁₋₄ haloalkyl.

In another aspect, the present invention relates to one of the following compounds, or a stereoisomer, a tautomer, an N-oxide, a solvate, a metabolite, a pharmaceutically acceptable salt or a prodrug thereof, but it is by no means limited to these compounds:

Error! Objects cannot be created from editing field codes. (1), Error! Objects cannot be created from editing field codes.

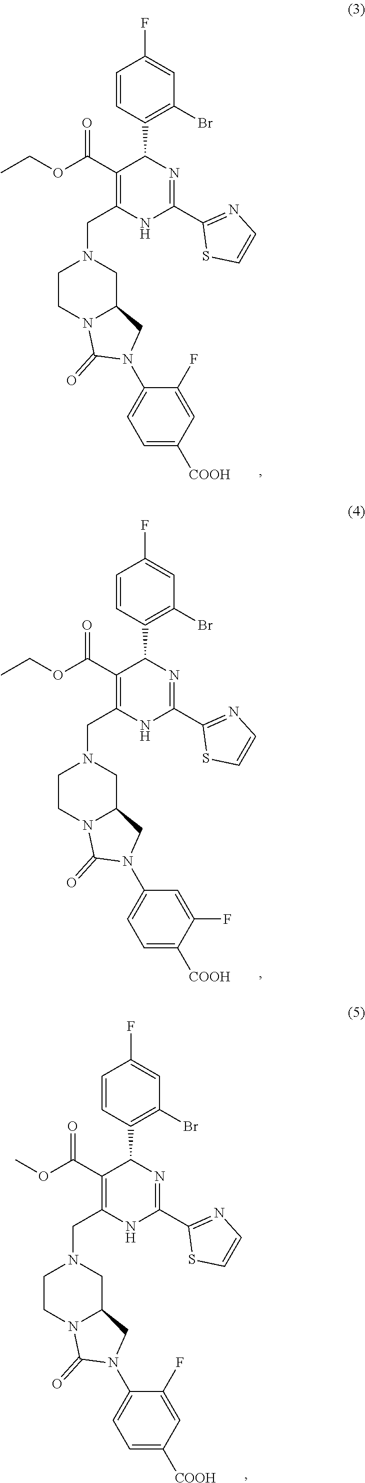 [SMILES: CCOC(=O)C1=C(CN2CCN3C(=O)N(c4ccc(C(=O)O)cc4F)C[C@@H]3C2)NC(c2nccs2)=N[C@H]1c1ccc(F)cc1Br ; CCOC(=O)C1=C(CN2CCN3C(=O)N(c4ccc(C(=O)O)c(F)c4)C[C@@H]3C2)NC(c2nccs2)=N[C@H]1c1ccc(F)cc1Br ; COC(=O)C1=C(CN2CCN3C(=O)N(c4ccc(C(=O)O)cc4F)C[C@@H]3C2)NC(c2nccs2)=N[C@H]1c1ccc(F)cc1Br]

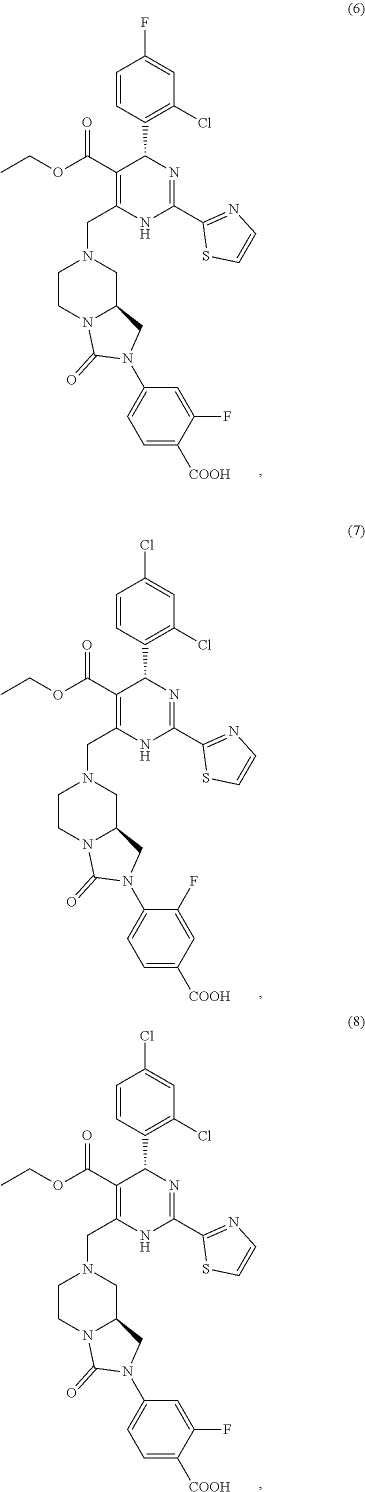 [SMILES: CCOC(=O)C1=C(CN2CCN3C(=O)N(c4ccc(C(=O)O)c(F)c4)C[C@@H]3C2)NC(c2nccs2)=N[C@H]1c1ccc(F)cc1Cl ; CCOC(=O)C1=C(CN2CCN3C(=O)N(c4ccc(C(=O)O)cc4F)C[C@@H]3C2)NC(c2nccs2)=N[C@H]1c1ccc(Cl)cc1Cl ; CCOC(=O)C1=C(CN2CCN3C(=O)N(c4ccc(C(=O)O)c(F)c4)C[C@@H]3C2)NC(c2nccs2)=N[C@H]1c1ccc(Cl)cc1Cl]

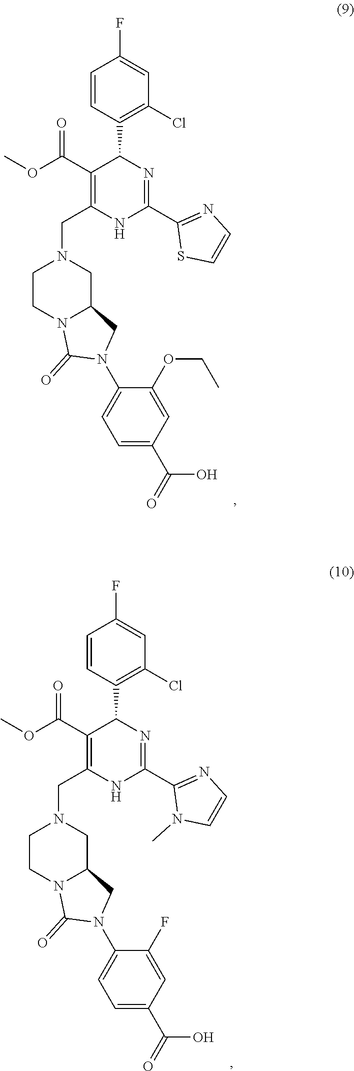 [SMILES: CCOc1cc(C(=O)O)ccc1N1C[C@@H]2CN(CC3=C(C(=O)OC)[C@H](c4ccc(F)cc4Cl)N=C(c4nccs4)N3)CCN2C1=O ; COC(=O)C1=C(CN2CCN3C(=O)N(c4ccc(C(=O)O)cc4F)C[C@@H]3C2)NC(c2nccn2C)=N[C@H]1c1ccc(F)cc1Cl]

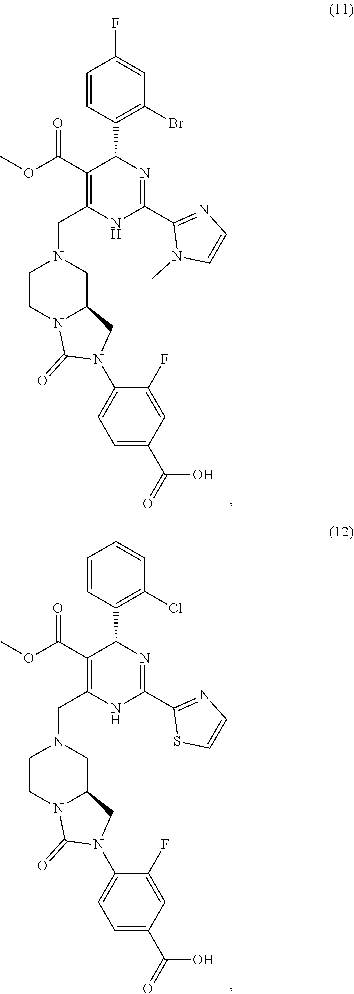 [SMILES: COC(=O)C1=C(CN2CCN3C(=O)N(c4ccc(C(=O)O)cc4F)C[C@@H]3C2)NC(c2nccn2C)=N[C@H]1c1ccc(F)cc1Br ; COC(=O)C1=C(CN2CCN3C(=O)N(c4ccc(C(=O)O)cc4F)C[C@@H]3C2)NC(c2nccs2)=N[C@H]1c1ccccc1Cl]

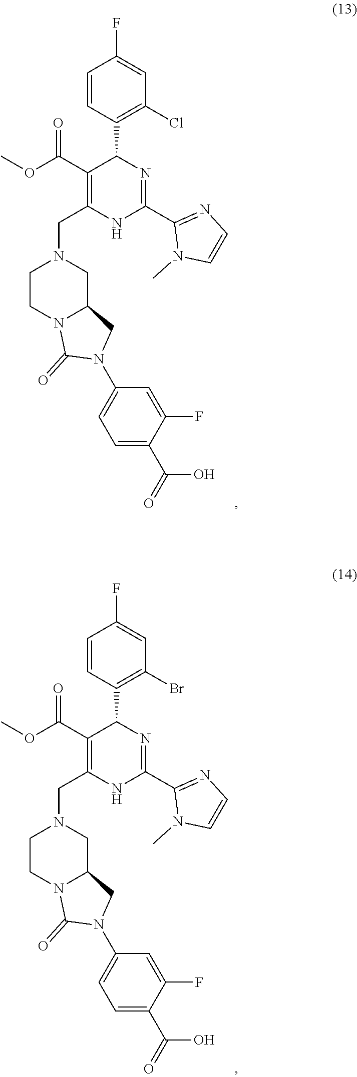 [SMILES: COC(=O)C1=C(CN2CCN3C(=O)N(c4ccc(C(=O)O)c(F)c4)C[C@@H]3C2)NC(c2nccn2C)=N[C@H]1c1ccc(F)cc1Cl ; COC(=O)C1=C(CN2CCN3C(=O)N(c4ccc(C(=O)O)c(F)c4)C[C@@H]3C2)NC(c2nccn2C)=N[C@H]1c1ccc(F)cc1Br]

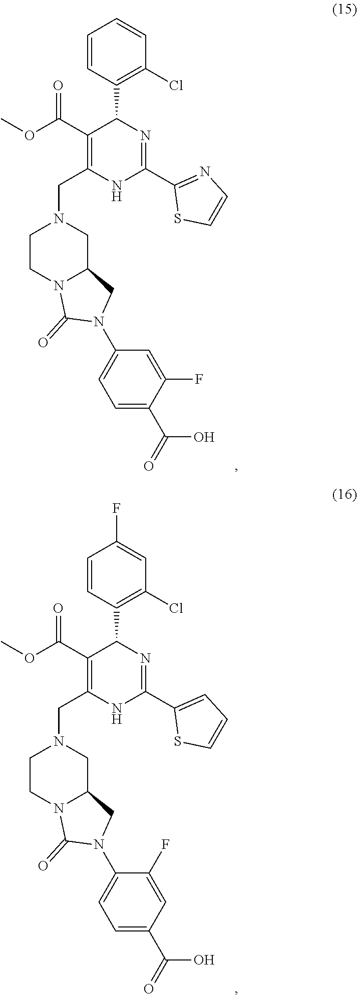 [SMILES: COC(=O)C1=C(CN2CCN3C(=O)N(c4ccc(C(=O)O)c(F)c4)C[C@@H]3C2)NC(c2nccs2)=N[C@H]1c1ccccc1Cl ; COC(=O)C1=C(CN2CCN3C(=O)N(c4ccc(C(=O)O)cc4F)C[C@@H]3C2)NC(c2cccs2)=N[C@H]1c1ccc(F)cc1Cl]

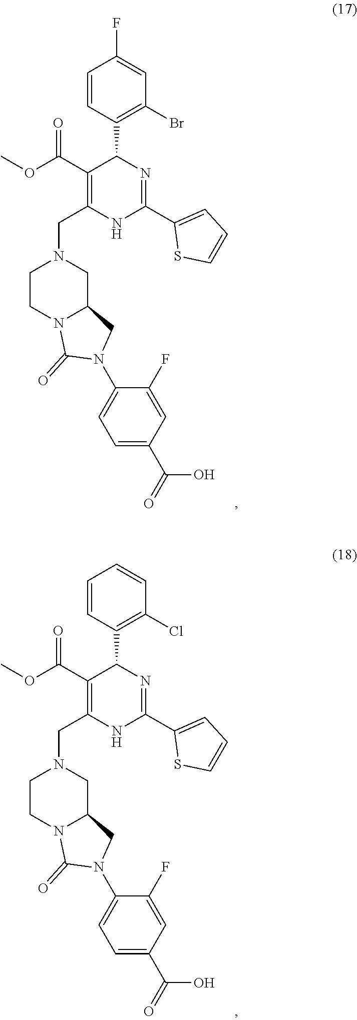 [SMILES: COC(=O)C1=C(CN2CCN3C(=O)N(c4ccc(C(=O)O)cc4F)C[C@@H]3C2)NC(c2cccs2)=N[C@H]1c1ccc(F)cc1Br ; COC(=O)C1=C(CN2CCN3C(=O)N(c4ccc(C(=O)O)cc4F)C[C@@H]3C2)NC(c2cccs2)=N[C@H]1c1ccccc1Cl]

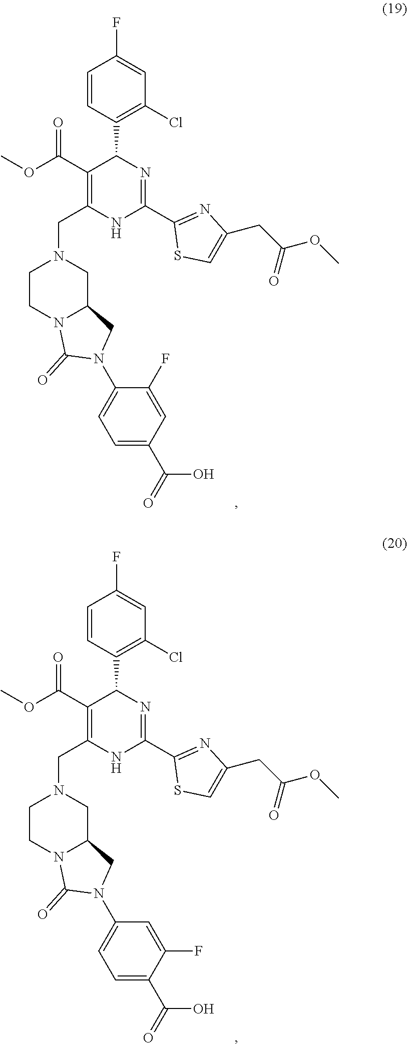

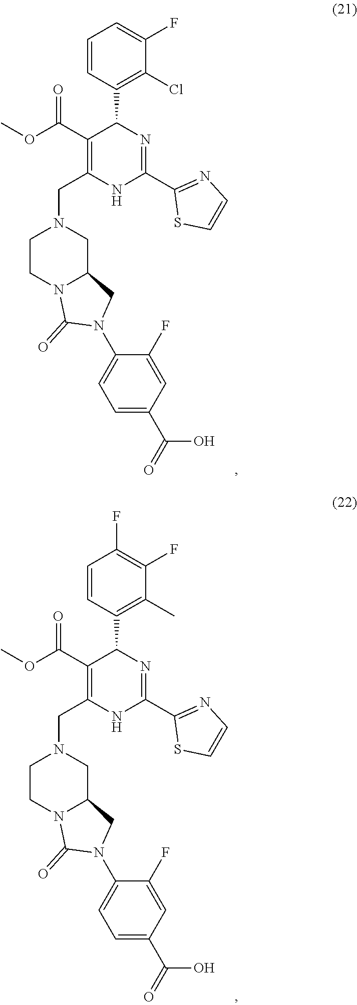 [SMILES: COC(=O)C1=C(CN2CCN3C(=O)N(c4ccc(C(=O)O)cc4F)C[C@@H]3C2)NC(c2nccs2)=N[C@H]1c1cccc(F)c1Cl ; COC(=O)C1=C(CN2CCN3C(=O)N(c4ccc(C(=O)O)cc4F)C[C@@H]3C2)NC(c2nccs2)=N[C@H]1c1ccc(F)c(F)c1C]

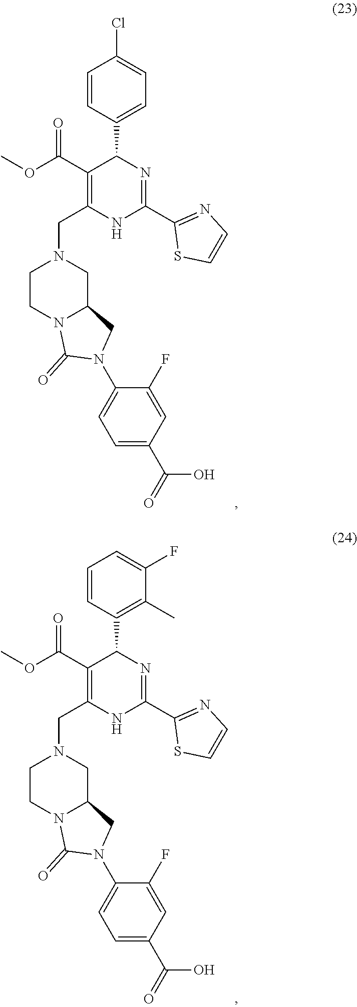 [SMILES: COC(=O)C1=C(CN2CCN3C(=O)N(c4ccc(C(=O)O)cc4F)C[C@@H]3C2)NC(c2nccs2)=N[C@H]1c1ccc(Cl)cc1 ; COC(=O)C1=C(CN2CCN3C(=O)N(c4ccc(C(=O)O)cc4F)C[C@@H]3C2)NC(c2nccs2)=N[C@H]1c1cccc(F)c1C]

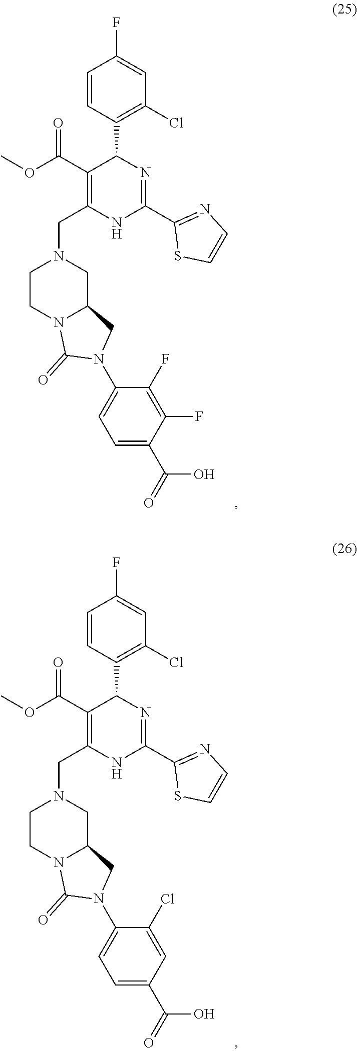 [SMILES: COC(=O)C1=C(CN2CCN3C(=O)N(c4ccc(C(=O)O)c(F)c4F)C[C@@H]3C2)NC(c2nccs2)=N[C@H]1c1ccc(F)cc1Cl ; COC(=O)C1=C(CN2CCN3C(=O)N(c4ccc(C(=O)O)cc4Cl)C[C@@H]3C2)NC(c2nccs2)=N[C@H]1c1ccc(F)cc1Cl]

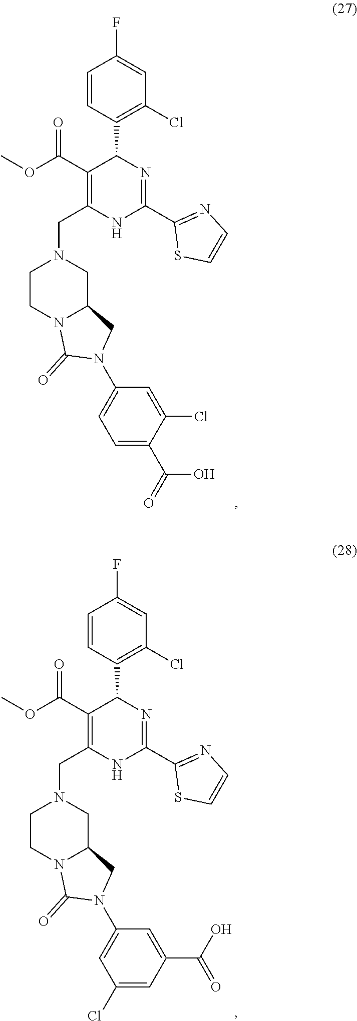 [SMILES: COC(=O)C1=C(CN2CCN3C(=O)N(c4ccc(C(=O)O)c(Cl)c4)C[C@@H]3C2)NC(c2nccs2)=N[C@H]1c1ccc(F)cc1Cl ; COC(=O)C1=C(CN2CCN3C(=O)N(c4cc(Cl)cc(C(=O)O)c4)C[C@@H]3C2)NC(c2nccs2)=N[C@H]1c1ccc(F)cc1Cl]

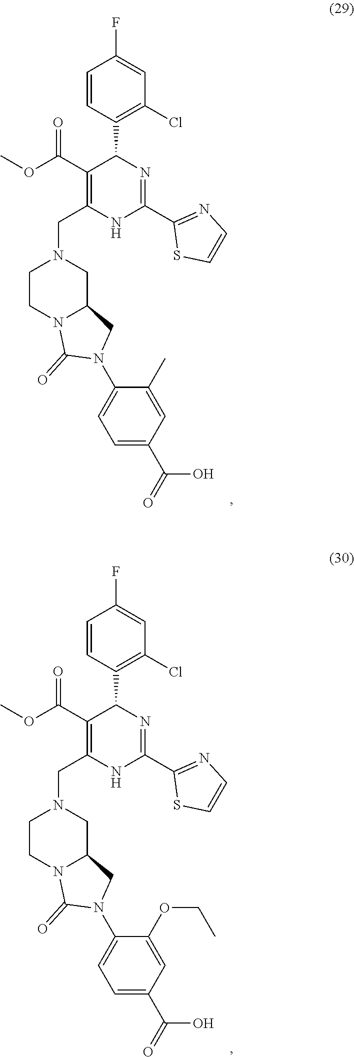 [SMILES: COC(=O)C1=C(CN2CCN3C(=O)N(c4ccc(C(=O)O)cc4C)C[C@@H]3C2)NC(c2nccs2)=N[C@H]1c1ccc(F)cc1Cl ; CCOc1cc(C(=O)O)ccc1N1C[C@@H]2CN(CC3=C(C(=O)OC)[C@H](c4ccc(F)cc4Cl)N=C(c4nccs4)N3)CCN2C1=O]

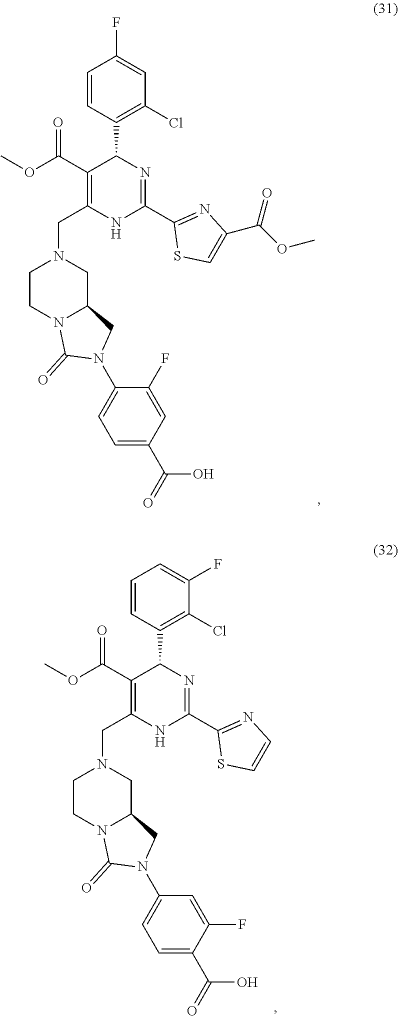

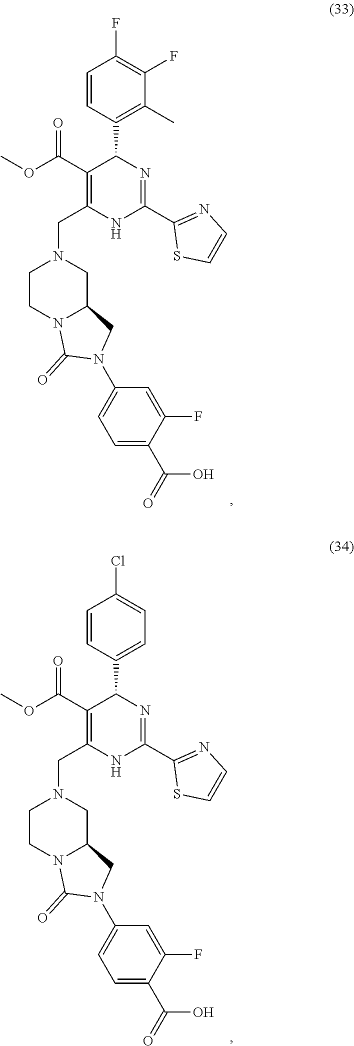 [SMILES: COC(=O)C1=C(CN2CCN3C(=O)N(c4ccc(C(=O)O)c(F)c4)C[C@@H]3C2)NC(c2nccs2)=N[C@H]1c1ccc(F)c(F)c1C ; COC(=O)C1=C(CN2CCN3C(=O)N(c4ccc(C(=O)O)c(F)c4)C[C@@H]3C2)NC(c2nccs2)=N[C@H]1c1ccc(Cl)cc1]

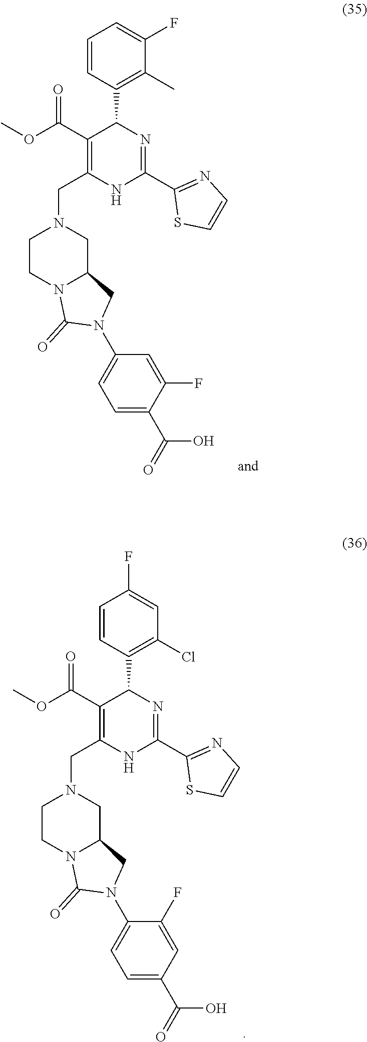 [SMILES: COC(=O)C1=C(CN2CCN3C(=O)N(c4ccc(C(=O)O)c(F)c4)C[C@@H]3C2)NC(c2nccs2)=N[C@H]1c1cccc(F)c1C ; COC(=O)C1=C(CN2CCN3C(=O)N(c4ccc(C(=O)O)cc4F)C[C@@H]3C2)NC(c2nccs2)=N[C@H]1c1ccc(F)cc1Cl]

In another aspect, provided herein is a pharmaceutical composition comprising the compound of the invention, and a pharmaceutically acceptable adjuvant.

In some embodiments, the pharmaceutical composition disclosed herein further comprises other anti-HBV drug.

In some embodiments of the pharmaceutical composition disclosed herein, wherein other anti-HBV drug is a HBV polymerase inhibitor, an immunomodulator or an interferon.

In some embodiments, wherein other anti-HBV drug is lamivudine, telbivudine, tenofovir, entecavir, adefovir dipivoxil, alfaferone, alloferon, celmoleukin, clevudine, emtricitabine, famciclovir, feron, hepatect CP, intefen, interferon α-1b, interferon α, interferon α-2a, interferon β-1a, interferon α-2, interleukin-2, mivotilate, nitazoxanide, peginterferon α-2a, ribavirin, roferon-A, sizofiran, Euforavac, rintatolimod, Phosphazid, Heplisav, interferon α-2b, levamisole, or propagermanium.

In other aspect, provided herein is use of the compound or the pharmaceutical composition disclosed herein in the manufacture a medicament for preventing, treating or lessening a virus disease in a patient.

In some embodiments of the use, the viral disease is hepatitis B infection or a disease caused by hepatitis B infection.

In other embodiments of the use, the disease caused by Hepatitis B infection is hepatic cirrhosis or hepatocellular carcinogenesis.

In other aspect, provided herein is the compound or the pharmaceutical composition disclosed herein for use in preventing, treating or lessening a viral disease in a patient.

In some embodiments, the viral disease is hepatitis B infection or a disease caused by hepatitis B infection.

In other embodiments, the disease caused by Hepatitis B infection is hepatic cirrhosis or hepatocellular carcinogenesis.

In another aspect, provided herein is a method of preventing, treating or lessening a viral disease in a patient, wherein the method comprises administering to a patient comprising administering to the patient a therapeutically effective amount of the compound or the pharmaceutical composition disclosed herein.

In some embodiments, wherein the viral disease is hepatitis B infection or a disease caused by hepatitis B infection.

In other embodiments, wherein the disease caused by Hepatitis B infection is hepatic cirrhosis or hepatocellular carcinogenesis.

In other aspect, provided herein is use of the compound or the pharmaceutical composition disclosed herein in the manufacture a medicament for preventing, treating or lessening Hepatitis B disease in a patient.

In other aspect, provided herein is a method of preventing, treating or lessening HBV disease in a patient, wherein the method comprises administering to a patient a pharmaceutically acceptable effective amount of the compound of the invention.

In other aspect, provided herein is a method of preventing, treating or lessening HBV disease in a patient, wherein the method comprises administering to a patient a pharmaceutically acceptable effective amount of a pharmaceutical composition containing the compound of the invention.

In other aspect, provided herein is use of the compound disclosed herein in the manufacture of a medicament for preventing, or treating HBV disease in a patient, and lessening the severity thereof.

In other aspect, provided herein is use of the pharmaceutical composition containing the compound disclosed herein in the manufacture of a medicament for preventing, or treating HBV disease in a patient, and lessening the severity thereof.

In some embodiments, the patient is a mammal, and in other embodiments, the patient is a human. In other embodiments, the use further comprises contacting the cell with an anti-HBV therapeutic agent.

In other aspect, provided herein is method of inhibiting HBV infection, the method comprises contacting a cell with a compound or pharmaceutical composition disclosed herein in a dose that can effectively inhibit HBV. In other embodiments, the method further comprises contacting the cell with another anti-HBV therapeutic agent.

In other aspect, provided herein is method of treating a patient with HBV disease, the method comprises administering a therapeutically effective amount of the compound disclosed herein or a pharmaceutical composition thereof to a patient in need.

In other embodiments, the method further comprises administering a therapeutically effective amount of other anti-HBV therapeutic agents to a patient in need.

In other aspect, provided herein is method of inhibiting HBV infection in a patient, the method comprises administering a therapeutically effective amount of the compound of the present invention or a pharmaceutical composition thereof to a patient in need. In other embodiments, the method further comprises administering a therapeutically effective amount of other anti-HBV therapeutic agent to a patient in need.

In other aspect, provided herein is method of preparing, separating and purifying the compound of Formula (I) or (Ia).

The present invention also relates to uses of the compound and pharmaceutically acceptable salts thereof in the manufacture of a medicament for effectively inhibiting HBV infection. The compound disclosed herein also can be used in the manufacture of a medicament for lessening, preventing, managing or treating a HBV disease in a patient.

Unless otherwise stated, all stereoisomers, geometric isomers, tautomers, N-oxides, hydrates, solvates, metabolites, pharmaceutically acceptable salts and prodrugs of the compounds disclosed herein are within the scope of the invention.

Specifically, the salt is a pharmaceutically acceptable salt. The phrase “pharmaceutically acceptable” refers to that the substance or composition must be compatible chemically and/or toxicologically, with the other ingredients comprising a formulation, and/or the mammal being treated therewith.

The compounds disclosed herein also include salts of the compounds which are not necessarily pharmaceutically acceptable salts, and which may be useful as intermediates for preparing and/or purifying compounds of Formula (I) or (Ia), and/or for separating enantiomers of compounds of Formula (I) or (Ia).

If the compound disclosed herein is a base, the desired salt may be prepared by any suitable method available in the art, for example, treatment of the free base with an inorganic acid, such as hydrochloric acid, hydrobromic acid, sulfuric acid, nitric acid, phosphoric acid, and the like. or with an organic acid, such as acetic acid, maleic acid, succinic acid, mandelic acid, fumaric acid, malonic acid, pyruvic acid, malic acid, 2-hydroxypropionic acid, citric acid, oxalic acid, glycolic acid and salicylic acid; a pyranosidyl acid, such as glucuronic acid and galacturonic acid; an alpha-hydroxy acid, such as citric acid and tartaric acid; an amino acid, such as aspartic acid and glutamic acid; an aromatic acid, such as benzoic acid and cinnamic acid; a sulfonic acid, such as p-toluenesulfonic acid, benzenesulfonic acid, methanesulfonic acid, ethanesulfonic acid, trifluoromethanesulfonic acid, and the like; or the combination thereof.

If the compound disclosed herein is an acid, the desired salt may be prepared by any suitable method, for example, treatment of the free acid with an inorganic or organic base, such as an amine (primary, secondary or tertiary), an alkali metal hydroxide, ammonium, N⁺(R¹⁴)₄ salt or alkaline earth metal hydroxide, and the like. Some non-limiting examples of suitable salts include organic salts derived from amino acids, such as glycine and arginine; ammonia, such as primary, secondary and tertiary amine, N⁺(R¹⁴)₄ salt, wherein R¹⁴ is H, C₁₋₄ alkyl, C₆₋₁₀ aryl, C₆₋₁₀ aryl-C₁₋₄-alkyl, and the like; and cyclic amines, such as piperidine, morpholine and piperazine, and inorganic salts derived from sodium, calcium, potassium, magnesium, manganese, iron, copper, zinc, aluminum, lithium, and the like, and further include, when appropriate, nontoxic ammonium, quaternary ammonium and amine cations formed using counterions such as halide, hydroxide, carboxylate, sulfate, phosphate, nitrate, C₁₋ₛ sulfonate or aryl sulfonate.

Pharmaceutical Compositions, Formulations, Administration and Uses of the Compounds and Pharmaceutical Compositions of the Present Invention

According to another aspect, the pharmaceutical composition of the invention comprises the compound of Formula (I) or (Ia). The compounds listed herein, or the compounds of the Examples, and a pharmaceutically acceptable adjuvant. The compound in the pharmaceutical composition disclosed herein can effectively inhibit hepatitis B virus, and is suitable for the treatment of virus-induced diseases, especially acute and chronic persistent HBV infection. Chronic viral diseases caused by HBV may lead to severe disease. Chronic hepatitis B virus infection can cause hepatic cirrhosis and/or hepatocellular carcinogenesis in many cases.

For the compound disclosed herein, the area of disease treatment that may be mentioned is, for example, the treatment of acute and chronic viral infections that may lead to infectious hepatitis, such as hepatitis B virus infection. The compound disclosed herein is particularly suitable for the treatment of chronic hepatitis B infection and acute and chronic hepatitis B virus infections.

The invention includes pharmaceutical formulation, in addition to non-toxic and inert pharmacologically suitable adjuvants, it also contains one or more compounds of Formula (I) or (Ia) or pharmaceutical compositions thereof or contains one or more active ingredients of the compound of Formula (I) or (Ia) or pharmaceutical compositions thereof.

The pharmaceutical formulations mentioned above may also contain other active pharmaceutical ingredients other than the compounds of Formula (I) or (Ia).

It will also be appreciated that certain of the compounds disclosed herein can exist in free form for treatment, or where appropriate, as a pharmaceutically acceptable derivative thereof. Some non-limiting examples of the pharmaceutically acceptable derivative include pharmaceutically acceptable prodrugs, salts, esters, salts of such esters, or any other adducts or derivatives which upon administration to a patient in need is capable of providing, directly or indirectly, a compound as otherwise described herein, or a metabolite or residue thereof.

As described above, the pharmaceutical compositions disclosed herein comprise any one of the compounds having Formula (I) or (Ia), and further comprise pharmaceutically acceptable adjuvants, such as those used herein, including any solvents, solid excipients, diluents, binders, disintegrants, or other liquid excipients, dispersion, corrigents or suspending agents, surfactants, isotonic agents, thickening agents, emulsifying agents, preservatives, solid binders, lubricants and the like, as suited to the particular dosage form desired. As described in the following: Troy et al., Remington: The Science and Practice of Pharmacy, 21st ed., 2005, Lippincott Williams & Wilkins, Philadelphia, and Swarbrick et al., Encyclopedia of Pharmaceutical Technology, eds. 1988-1999, Marcel Dekker, New York, incorporated herein by reference in their entireties, discloses various excipients used in formulating pharmaceutically acceptable compositions and known techniques for the preparation thereof. Except insofar as any conventional excipients incompatible with the compounds disclosed herein, such as by producing any undesirable biological effect or otherwise interacting in a deleterious manner with any other components of the pharmaceutically acceptable composition, its use is contemplated to be within the scope of this invention.

Some non-limiting examples of materials which can serve as pharmaceutically acceptable excipients include ion exchangers; aluminium; aluminum stearate; lecithin; serum proteins such as human serum albumin; buffer substances such as phosphates; glycine; sorbic acid; potassium sorbate; partial glyceride mixtures of saturated vegetable fatty acids; water; salts or electrolytes such as protamine sulfate, disodium hydrogen phosphate, potassium hydrogen phosphate, sodium chloride and zinc salts; colloidal silica; magnesium trisilicate; polyvinyl pyrrolidone; polyacrylates; waxes; polyethylene-polyoxypropylene-block polymers; wool fat; sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol and polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants.

The pharmaceutical composition of the compound disclosed herein can be administered in any of the following ways: orally, inhaled by spray, locally, rectally, nasally, locally, vaginally, parenterally such as subcutaneous, intravenous, intramuscular, intraperitoneal, intrathecal, intraventricular, intrasternal, or intracranial injection or infusion, or administered with the aid of an explanted reservoir. The preferred modes of administration are administered orally, intramuscularly, intraperitoneally or intravenously.

The compounds or pharmaceutical compositions thereof disclosed herein can be administered in a unit dosage form. The dosage form may be in a liquid form, or a solid form. The liquid form includes true solutions, colloids, particulates, suspensions. Other dosage forms include tablets, capsules, dropping pills, aerosols, pills, powders, solutions, suspensions, emulsions, granules, suppositories, freeze-dried powder injection, clathrates, implants, patches, liniments, and the like.

Oral tablets and capsules may comprise excipients, e.g., binders, such as syrup, Arabic gum, sorbitol, tragacanth or polyvinylpyrrolidone; fillers, such as lactose, sucrose, corn starch, calcium phosphate, sorbitol, glycine; lubricants such as magnesium stearate, talc, polyethylene glycol, silica; disintegrating agents, such as potato starch, or acceptable moisturizing agents such as sodium lauryl sulfate. Tablets may be coated by using known methods in pharmaceutics.

Oral solution may be made as a suspension of water and oil, a solution, an emulsion, syrup or an elixir, or made as a dried product to which water or other suitable medium is added before use. This liquid preparation may comprise conventional additives, e.g., suspending agents such sorbitol, cellulose methyl ether, glucose syrup, gel, hydroxyethyl cellulose, carboxymethyl cellulose, aluminum stearate gel, hydrogenated edible greases; emulsifying agents such as lecithin, sorbitan monoleate, Arabic gum; or non-aqueous adjuvant (possibly including edible oil), such as almond oil, grease such as glycerin, ethylene glycol, or ethanol; antiseptics such as methyl or propyl p-hydroxybenzoate, sorbic acid. If desired, a flavoring agent or a colorant may be added.

Suppositories may comprise a conventional suppository base, such as cocoa butter or other glyceride.

For parenteral administration, the liquid dosage form is usually made from the compound and a sterilized adjuvant. Water is the preferred adjuvant. According to the difference of selected adjuvant and drug concentration, the compound can be either dissolved in the adjuvant or made into a supernatant solution. When being made into a solution for injection, the compound is firstly dissolved in water, and then filtered and sterilized before being packaged into a sealed bottle or an ampoule.

For application topically to the skin, the compound disclosed herein may be made into a suitable form of ointments, lotions or creams, wherein the active ingredient is suspended or dissolved in one or more adjuvant(s). Wherein adjuvants used for an ointment preparation include, but are not limited to: mineral oil, liquid vaseline, white vaseline, propylene glycol, polyoxyethylene, polyoxypropylene, emulsified wax and water; adjuvants used for a lotion and a cream include, but are not limited to: mineral oil, sorbitan monostearate, Tween 60, cetyl ester wax, hexadecylene aromatic alcohol, 2-octyl dodecanol, benzyl alcohol and water.

In general, it has proved to be advantageous in either human medicine or veterinary medicine, the total administrated dose of the active compound disclosed herein is about 0.5 to 500 mg every 24 hours, preferably 1 to 100 mg/kg body weight. If appropriate, the drug is administrated in single dose for multiple times, to achieve the desired effect. The amount of the active compound in a single dose is preferably about 1 to 80 mg, more preferably 1 to 50 mg/kg body weight. Nevertheless, the dose may also be varied according to the kind and the body weight of treatment objects, the nature and the severity of diseases, the type of preparations and the method of administration of drugs, and administration period or time interval.

The pharmaceutical composition provided herein further comprises anti-HBV drugs. Wherein the anti-HBV drug is a HBV polymerase inhibitor, an immunomodulator or an interferon.

The HBV agent is lamivudine, telbivudine, tenofovir, entecavir, adefovir dipivoxil, alfaferone, alloferon, celmoleukin, clevudine, emtricitabine, famciclovir, feron, hepatect CP, intefen, interferon α-1b, interferon α, interferon α-2a, interferon β-1a, interferon α-2, interleukin-2, mivotilate, nitazoxanide, peginterferona-2a, ribavirin, roferon-A, sizofiran, euforavac, veldona, rintatolimod, phosphazid, heplisav, interferon α-2b, levamisole, or propagermanium, and the like.

In other aspect, provided herein is use of the compound or the pharmaceutical composition disclosed herein in the manufacture of a medicament for preventing, treating or lessening Hepatitis B disease in a patient, including administering to the patient a pharmaceutically acceptable effective dose. Hepatitis B disease refers to liver diseases caused by hepatitis B virus infection or hepatitis B infection, including acute hepatitis, chronic hepatitis, hepatic cirrhosis and hepatocellular carcinogenesis. Acute hepatitis B virus infection can be asymptomatic or manifest as acute hepatitis symptoms. Patients with chronic viral infections have active diseases that can progress to hepatic cirrhosis and liver cancer.

The anti-HBV drug can be administered separately from the pharmaceutical composition comprising the compound of the present invention, as a part of the multi-administration regimen. Alternatively, those therapeutic agents may be part of a single dosage form, mixed with the compound of the present invention to form a single composition. If the administration is part of a multi-dosing regimen, the two active agents can be delivered simultaneously or continuously for a period of time to obtain the target reagent activity.

The amount of compound and composition (those containing a composition as described in the present invention) that can be combined with the excipient to produce a single dosage form varies depending on the main treatment and the particular mode of administration. Normally, the amount of the composition of the present invention will not exceed the amount of normal administration of the composition comprising as the sole active agent. On the other hand, the range of the amount of the presently disclosed composition is about 50%-100% of the normal amount of the existing composition, and the contained agent is used as the sole active therapeutic agent. Among those included compositions, the composition will act synergistically with the compound provided herein.

The compound provided herein shows a strong antiviral effect. Such compounds have unexpected antiviral activity against HBV, so they are suitable for the treatment of various diseases caused by viruses, especially those caused by acute and chronic persistent HBV viral infections. Chronic viral diseases caused by HBV can cause various syndromes of varying severity. It is well known that chronic hepatitis B virus infection can cause liver cirrhosis and/or hepatocellular carcinoma.

Examples of indications that can be treated with the compounds of the present invention are: acute and chronic viral infections that can lead to infectious hepatitis, such as hepatitis B virus infection. Particularly preferred are chronic hepatitis B infection and acute hepatitis B virus infection.

The present invention also relates to use of the compound and pharmaceutical composition disclosed herein in the manufacture of a medicament for treating and preventing viral diseases, especially hepatitis B.

### General Synthetic Procedures

Generally, the compounds disclosed herein may be prepared by methods described herein, wherein the substituents are as defined for Formula (I) or (Ia) above, except where further noted. The following non-limiting synthesis schemes and examples are presented to further exemplify the invention.

Persons skilled in the art will recognize that the chemical reactions described may be readily adapted to prepare a number of other compounds disclosed herein, and alternative methods for preparing the compounds disclosed herein are deemed to be within the scope disclosed herein. For example, the synthesis of non-exemplified compounds according to the invention may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds disclosed herein.

In the examples described below, unless otherwise indicated all temperatures are set forth in degrees Celsius (° C.). Reagents were purchased from commercial suppliers such as Aldrich Chemical Company, Arco Chemical Company and Alfa Chemical Company, and were used without further purification unless otherwise indicated. Common solvents were purchased from commercial suppliers such as Shantou XiLong Chemical Factory, Guangdong Guanghua Reagent Chemical Factory Co. Ltd., Guangzhou Reagent Chemical Factory, Tianjin YuYu Fine Chemical Ltd., Qingdao Tenglong Reagent Chemical Ltd., and Qingdao Ocean Chemical Factory.

Column chromatography was conducted using a silica gel column. Silica gel (200-300 mesh) was purchased from Qingdao Ocean Chemical Factory. NMR spectroscopy were obtained by using CDCl₃, DMSO-d₆, CD₃OD or acetone-d₆ as solvents (reported in ppm), with TMS (0 ppm) or chloroform (7.25 ppm) as the reference standard. When peak multiplicities were reported, the following abbreviations were used: s (singlet), d (doublet), t (triplet), m (multiplet), br (broadened), dd (doublet of doublets), dt (doublet of triplets), and br.s (broadened singlet). Coupling constants J, when given, were reported in Hertz (Hz).

Low-resolution mass spectral (MS) data were also determined on an Agilent 6320 series LC-MS spectrometer equipped with G1312A binary pumps, a G1316A TCC (Temperature Control of Column, maintained at 30° C.), a G1329A autosampler and a G1315B DAD detector were used in the analysis. An ESI source was used on the LC-MS spectrometer.

Low-resolution mass spectral (MS) data were also determined on an Agilent 6120 series LC-MS spectrometer equipped with G1311A binary pumps, a G1316A TCC (Temperature Control of Column, maintained at 30° C.), a G1329A autosampler and a G1315D DAD detector were used in the analysis. An ESI source was used on the LC-MS spectrometer.

Both LC-MS spectrometers were equipped with an Agilent Zorbax SB-C18, 2.1×30 mm, 5 μm column. Injection volume was decided by the sample concentration. The flow rate was 0.6 mL/min. The HPLC peaks were recorded by UV-Vis wavelength at 210 nm and 254 nm. The mobile phase was 0.1% formic acid in acetonitrile (phase A) and 0.1% formic acid in ultrapure water (phase B). The gradient elution conditions were showed in Table 1:

TABLE 1
Gradient elution conditions

| Time (min) | A (CH₃CN, 0.1% HCOOH) | B (H₂O, 0.1% HCOOH) |
|---|---|---|
| 0-3 | 5-100 | 95-0 |
| 3-6 | 100 | 0 |
| 6-6.1 | 100-5 | 0-95 |
| 6.1-8 | 5 | 95 |

Purities of compounds were assessed by Agilent 1100 Series high performance liquid chromatography (HPLC) with UV detection at 210 nm and 254 nm (Zorbax SB-C18, 2.1×30 mm, 4 micron). The run time was 10 min, and the flow rate was 0.6 mL/min. The elution was performed with a gradient of 5 to 95% phase A (0.1% formic acid in CH3CN) in phase B (0.1% formic acid in H2O). Column was operated at 40° C.

The following abbreviations are used throughout the specification:

| | | | |
|---|---|---|---|
| MeOH | methanol | Na₂SO₄ | sodium sulfate |
| McOH-d₄ | deuterated methanol | Et3N, TEA | triethylamine |
| DCM, CH₂Cl₂ | dichloromethane | H2O | water |
| CHCl₃ | chloroform, Trichloromethane | mL, ml | milliliter |
| CDCl₃ | chloroform-d | RT, rt | room temperature |
| Ph₃P | triphenylphosphine | Rt | retention time |
| LiOH•H₂O | Lithium hydroxide monohydrate | 1 atm | 101.325 kPa |
| tBu XPhos | 2-di-t-butylphosphino-2′,4′,6′-tri-i-propyl-1,1′- | h | hour, hours |
| | biphenyl | | |
| TFA | trifluoroacetic acid | H₂ | hydrogen |
| (dppf)PdCl₂ | [1,1′-bis(diphenylphosphino)ferrocene]dichloropalladium(II) | HCl/EA, HCl/EtOAc | Hydrogen chloride in ethyl acetate |
| (dppf)PdCl₂•CH₂Cl₂ | 1,1′-bis(diphenylphosphino)ferrocene-palladium(II)dichloride | HOAt | 1-hydroxy-7-azabenzotriazole |
| | dichloromethane complex | | |
| X-PHOS, X-Phos | 2-(dicyclohexylphosphino)-2′,4′,6′-tri-i-propyl-1,1′- | HATU | O-(7-Azabenzotriazol-l-yl)-N,N,N′,N′- |
| | biphenyl | | tetramethylurea hexafluorophosphate |
| Xantphos | dimethylbisdiphenylphosphinoxanthene | DIPEA | N,N-diisopropylethylamine |
| Pd/C | Palladium on activated carbon | DMF | N,N-dimethylformamide |
| Boc | tert-butoxycarbonyl | THF | tetrahydrofuran |
| (Boc)₂O | Di-tert-butyl dicarbonate | DMSO | dimethylsulfoxide |
| Pd₂(dba)₃ | tris(dibenzylideneacetone)dipalladium | CH₃OH | methanol |
| CH₃I | Methyl iodide | N2 | nitrogen |
| SOCl₂ | Thionyl chloride | NH₄Cl | ammonium chloride |
| PE | petroleum ether | Ac₂O | acetic anhydride |
| EtOAc, EA | ethyl acetate | t_{1/2} | half-life |
| EtOH | ethyl alcohol | AUC | area under the drug-time curve |
| HCl | hydrochloric acid | Vss | steady-state apparent volume of |
| | | | distribution |
| K₂CO₃ | potassium carbonate | CL | clearance |
| NaHCO₃ | sodium bicarbonate | F | absolute bioavailability |
| NaOH | sodium hydroxide | Dose | dosage |
| NaCl | sodium chloride | Tₘₐₓ | peak time |
| | | Cₘₐₓ | maximum concentration |
| | | Hr*ng/mL | blood concentration*time |

### Synthetic Scheme

The following synthetic scheme lists the experimental procedures for preparing the compounds disclosed herein. wherein each of R¹, R², R³, R^{1a}, R^{1b}, R⁴, R^{4a}, R^{4b} and R^{y} is as defined herein.

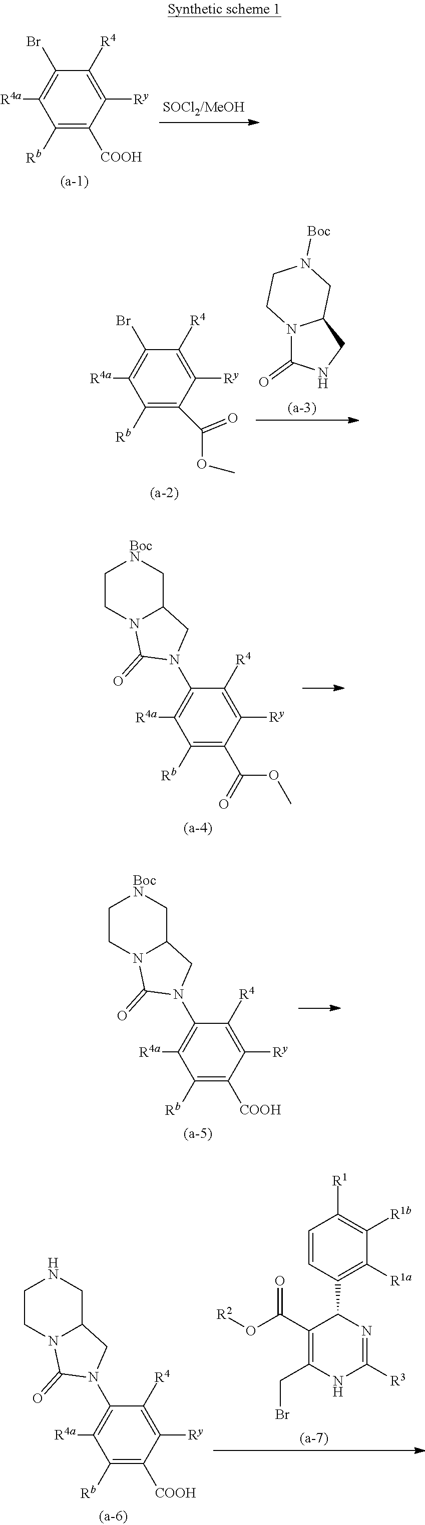

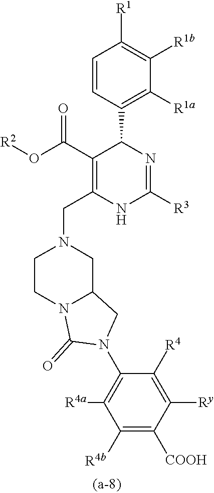

Compound (a-8) disclosed herein can be prepared by the process illustrated in Synthetic scheme 1. First, compound (a-1) is reacted with thionyl chloride and methanol to obtain compound (a-2). Then, compound (a-2) and compound (a-3) are reacted under alkaline condition (such as cesium carbonate, etc.) with a catalyst (such as palladium acetate, etc.), a ligand (such as X-PHOS, t-BuX-PHOS, etc.) and a suitable solvent (such as 1,4-dioxane, etc.) to obtain compound (a-4). Next, compound (a-4) undergoes an ester hydrolysis reaction under alkaline condition (such as lithium hydroxide aqueous solution, etc.) to obtain compound (a-5), and then Boc is removed to obtain compound (a-6). Finally, compound (a-6) or its salt and compound (a-7) (compound (a-7) can be prepared by referring to the synthesis scheme 1 in WO2015074546 and the specific example methods therein) are reacted under alkaline condition (such as potassium carbonate, etc.) with a suitable solvent (such as ethanol, etc.) to obtain the compound (a-8).

### EXAMPLES

The following examples are used to illustrate the present invention, but not to limit the scope of the present invention.

### Examples

In the following preparation examples, the inventors described the preparation process of the compounds disclosed herein in detail by taking part of the compounds as examples.

Synthesis of Fragment F1:

Error! Objects cannot be created from editing field codes.

Synthesis of F1-1:

F1-0 (3 g, 13.7 mmol) was dissolved in methanol (30 mL), thionyl chloride (1.2 mL, 16 mmol) was slowly added dropwise under ice bath. After the addition, the mixture was reacted at room temperature for 12 h. After the reaction, the solvent was evaporated under reduced pressure. Petroleum ether (90 mL) was added to the residue for extraction, and the organic layer was washed with saturated sodium bicarbonate aqueous solution (30 mL×3) and saturated sodium chloride solution (30 mL) successively. Then the mixture was dried over anhydrous sodium sulfate and was concentrated under reduced pressure to obtain F1 as colorless oil (3.05 g, 95.6%).

Synthesis of F1-2:

F1-1 (3.05 g, 13.1 mmol), tert-butyl (R)-3-oxohexahydroimidazo[1,5-a]pyrazine-7(1H)-carboxylate (2.9 g, 12 mmol), Pd₂(dba)₃ (0.55 g, 0.60 mmol), Xantphos (0.70 g, 1.2 mmol), cesium carbonate (5.9 g, 18 mmol) and 1,4-dioxane (30 mL) were added to a dried reaction flask in turn. The mixture was stirred for 24 h under nitrogen protection at 100° C., then the mixture was filtered with diatomaceous earth to obtain a filter cake. The filter cake was washed with dichloromethane (200 mL), and the filtrate was spin-dried. The residue was separated and purified by silica gel column chromatography (PE/EA (V/V)=2/1) to obtain F1-2 as a white solid (2.5 g, 53%). MS (ESI, pos. ion) m/z: 416.2 [M+Na]⁺.

Synthesis of F1-3:

F1-2 (2.5 g, 6.35 mmol), methanol (10 mL), tetrahydrofuran (20 mL) and lithium hydroxide monohydrate (0.8 g, 19 mmol) were added to a dried reaction flask in turn, and the mixture was reacted at 50° C. for 12 h. Then the mixture was concentrated under reduced pressure. The residue was diluted with water (100 mL), 1M hydrochloric acid was used to adjust pH to 4-5, and the residue was filtered to obtain F1-3 as a white solid (2.31 g, 95.8%). MS (ESI, pos. ion) m/z: 402.2 [M+Na]⁺.

Synthesis of F1:

F1-3 (300 mg, 0.79 mmol) was dissolved in dichloromethane (2 mL), then trifluoroacetic acid (2 mL) was added. The mixture was stirred at room temperature for 0.5 h, and then the mixture was concentrated under reduced pressure to obtain F1 as brown oil (311 mg, 100%). MS (ESI, pos. ion) m/z: 280.0 [M+H]⁺.

Synthesis of Fragment F2:

Error! Objects cannot be created from editing field codes.

F1-0 was replaced by F2-0, and the rest of the experimental operation referred to the synthesis method of fragment F1, and F2 was obtained as brown oil.

### Synthesis of Example 1

Error! Objects cannot be created from editing field codes.

F1 (311 mg, 0.79 mmol), (R)-6-(bromomethyl)-4-(2-chloro-4-fluorophenyl)-2-(thiazol-2-yl)-1,4-dihydropyrimidine-5-carboxylate (350 mg, 0.79 mmol), ethanol (10 mL) and potassium carbonate (0.276 g, 2 mmol) were added to a dried reaction flask in turn, the mixture was stirred and reacted at room temperature for 12 h. After the reaction, water (50 mL) was added to dilute the mixture, 1M hydrochloric acid was added to adjust pH to 5-6, then dichloromethane (50 mL×3) was added to extract the mixture, and the combined organic phase was concentrated under reduced pressure to obtain a residue. The obtained residue was separated and purified by silica gel column chromatography (DCM/CH₃OH (V/V)=50/1) to obtain the title compound as a yellow solid (155 mg, 30.5%). MS (ESI, pos. ion) m/z: 643.3 [M+H]⁺; ¹H NMR (400 MHz, DMSO-d₆) δ (ppm): 9.70 (s, 1H), 8.03 (d, J=3.0 Hz, 1H), 7.94 (d, J=3.0 Hz, 1H), 7.80-7.73 (m, 2H), 7.70 (d, J=12.0 Hz, 1H), 7.46-7.37 (m, 2H), 7.18 (td, J=8.5, 2.3 Hz, 1H), 6.05 (s, 1H), 4.08-4.00 (m, 1H), 4.00-3.91 (m, 2H), 3.90-3.78 (m, 2H), 3.58 (dd, J=8.6, 4.1 Hz, 1H), 3.52 (s, 3H), 3.13-3.05 (m, 1H), 2.99-2.92 (m, 2H), 2.38-2.27 (m, 1H), 2.22 (t, J=10.8 Hz, 1H).

### Synthesis of Example 2

Error! Objects cannot be created from editing field codes.

F2 (311 mg, 0.79 mmol), methyl (R)-6-(bromomethyl)-4-(2-chloro-4-fluorophenyl)-2-(thiazol-2-yl)-1,4-dihydropyrimidine-5-carboxylate (350 mg, 0.79 mmol), ethanol (10 mL) and potassium carbonate (0.276 g, 2 mmol) were added to a dried reaction flask in turn, the mixture was stirred and reacted at room temperature for 12 h. After the reaction, water (50 mL) was added to dilute the reaction mixture, and 1M hydrochloric acid was added to adjust pH to 5-6, then dichloromethane (50 mL×3) was added to extract the reaction mixture, and the combined organic phase was concentrated under reduced pressure to obtain a residue. The obtained residue was separated and purified by silica gel column chromatography (DCM/CH₃OH (V/V)=50/1) to obtain the titled compound as a yellow solid (260 mg, 51.13%). MS (ESI, pos. ion) m/z: 643.2 [M+H]⁺; ¹H NMR (400 MHz, CDCl₃) δ (ppm): 9.60 (s, 1H), 7.98 (t, J=8.6 Hz, 1H), 7.87 (d, J=3.1 Hz, 1H), 7.56 (d, J=13.7 Hz, 1H), 7.49 (d, J=3.1 Hz, 1H), 7.36-7.29 (m, 2H), 7.15 (dd, J=8.5, 2.5 Hz, 1H), 6.94 (td, J=8.3, 2.5 Hz, 1H), 6.22 (s, 1H), 4.16 (d, J=17.0 Hz, 1H), 4.12-4.01 (m, 2H), 3.97-3.89 (m, 2H), 3.62 (s, 3H), 3.45 (dd, J=9.2, 4.9 Hz, 1H), 3.30 (td, J=13.0, 3.1 Hz, 1H), 2.96 (t, J=10.5 Hz, 2H), 2.53 (td, J=11.3, 2.9 Hz, 1H), 2.28 (t, J=10.6 Hz, 1H).

### Synthesis of Example 3

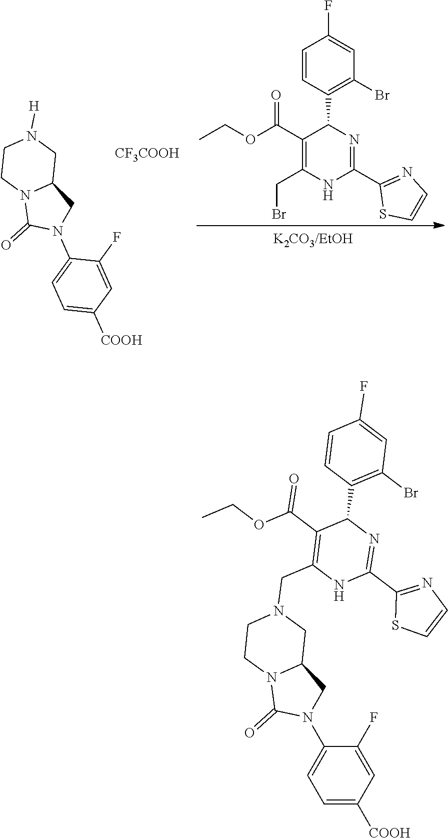 [SMILES: O=C(O)c1ccc(N2C[C@@H]3CNCCN3C2=O)c(F)c1 ; "CF3COOH" ; CCOC(=O)C1=C(CBr)NC(c2nccs2)=N[C@H]1c1ccc(F)cc1Br ; CCOC(=O)C1=C(CN2CCN3C(=O)N(c4ccc(C(=O)O)cc4F)C[C@@H]3C2)NC(c2nccs2)=N[C@H]1c1ccc(F)cc1Br]

Methyl (R)-6-(Bromomethyl)-4-(2-chloro-4-fluorophenyl)-2-(thiazol-2-yl)-1,4-dihydropyrimidine-5-carboxylate of Example 1 was replaced with ethyl (R)-4-(2-bromo-4-fluorophenyl)-6-(bromomethyl)-2-(thiazol-2-yl)-1,4-dihydropyrimidine-5-carboxylate (0.3 g, 0.6 mmol), and the rest were operated according to the method of Example 1 to obtain a yellow solid (0.24 g, 56.0%). MS (ESI, pos. ion) m/z: 701.1 [M+H]; ¹H NMR (400 MHz, CDCl₃) δ (ppm): 9.60 (s, 1H), 7.94-7.75 (m, 4H), 7.48 (d, J=3.1 Hz, 1H), 7.36-7.29 (m, 2H), 6.99 (td, J=8.3, 2.5 Hz, 1H), 6.22 (s, 1H), 4.22-3.92 (m, 7H), 3.65 (d, J=3.2 Hz, 1H), 3.35-3.25 (m, 1H), 2.95-2.89 (m, 2H), 2.58-2.53 (m, 1H), 2.35 (d, J=9.2 Hz, 1H), 1.15 (t, J=7.1 Hz, 3H).

### Synthesis of Example 4

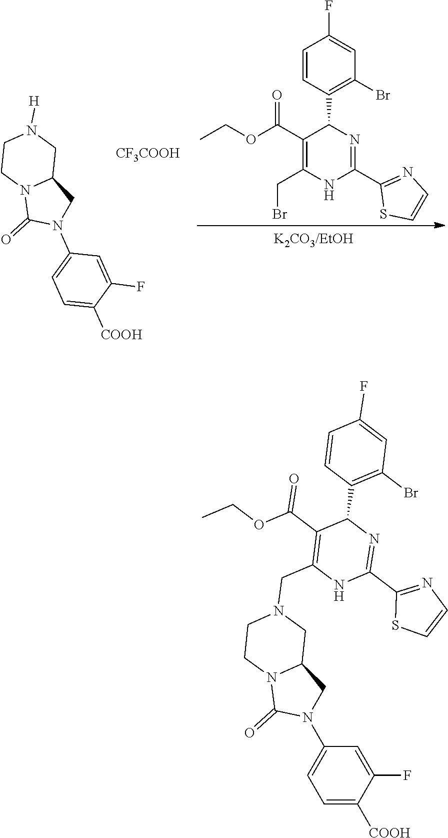 [SMILES: O=C(O)c1ccc(N2C[C@@H]3CNCCN3C2=O)cc1F ; "CF3COOH" ; CCOC(=O)C1=C(CBr)NC(c2nccs2)=N[C@H]1c1ccc(F)cc1Br ; CCOC(=O)C1=C(CN2CCN3C(=O)N(c4ccc(C(=O)O)c(F)c4)C[C@@H]3C2)NC(c2nccs2)=N[C@H]1c1ccc(F)cc1Br]

Methyl (R)-6-(Bromomethyl)-4-(2-chloro-4-fluorophenyl)-2-(thiazol-2-yl)-1,4-dihydropyrimidine-5-carboxylate of Example 2 was replaced with ethyl (R)-4-(2-bromo-4-fluorophenyl)-6-(bromomethyl)-2-(thiazol-2-yl)-1,4-dihydropyrimidine-5-carboxylate (0.3 g, 0.6 mmol), and the rest were operated according to the method of Example 2 to obtain a yellow solid (84 mg, 20.0%). MS (ESI, pos. ion) m/z: 701.1 [M+H]⁺; ¹H NMR (400 MHz, CDCl₃) δ (ppm): 9.55 (s, 1H), 7.99 (t, J=8.5 Hz, 1H), 7.86 (d, J=3.0 Hz, 1H), 7.56 (d, J=13.8 Hz, 1H), 7.48 (d, J=3.0 Hz, 1H), 7.39-7.29 (m, 3H), 6.99 (td, J=8.4, 2.3 Hz, 1H), 6.22 (s, 1H), 4.21-3.92 (m, 7H), 3.45 (dd, J=9.0, 4.9 Hz, 1H), 3.35-3.25 (m, 1H), 3.02-2.92 (m, 2H), 2.57-2.48 (m, 1H), 2.27 (t, J=10.6 Hz, 1H), 1.15 (t, J=7.1 Hz, 3H).

### Synthesis of Example 5

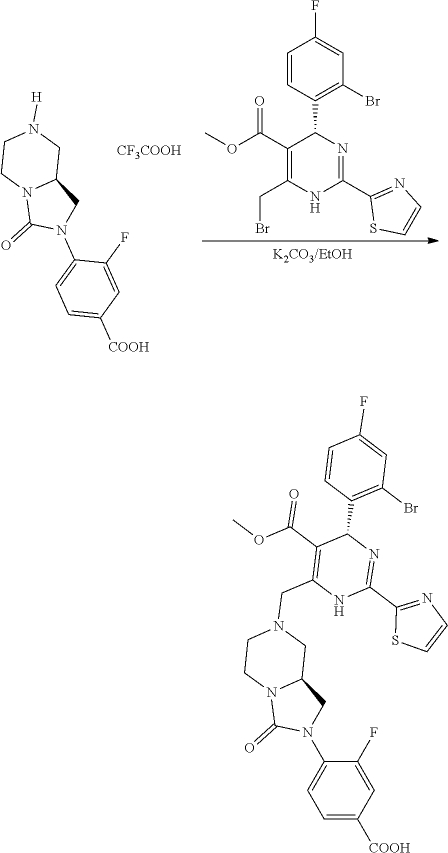 [SMILES: O=C(O)c1ccc(N2C[C@@H]3CNCCN3C2=O)c(F)c1 ; "CF3COOH" ; COC(=O)C1=C(CBr)NC(c2nccs2)=N[C@H]1c1ccc(F)cc1Br ; COC(=O)C1=C(CN2CCN3C(=O)N(c4ccc(C(=O)O)cc4F)C[C@@H]3C2)NC(c2nccs2)=N[C@H]1c1ccc(F)cc1Br]

Methyl (R)-6-(Bromomethyl)-4-(2-chloro-4-fluorophenyl)-2-(thiazol-2-yl)-1,4-dihydropyrimidine-5-carboxylate of Example 1 was replaced with methyl (R)-4-(2-Bromo-4-fluorophenyl)-6-(bromomethyl)-2-(thiazol-2-yl)-1,4-dihydropyrimidine-5-carboxylate (0.3 g, 0.6 mmol), and the rest were operated according to the method of Example 1 to obtain a yellow solid (0.3 g, 73%). MS (ESI, pos. ion) m/z: 687.0 [M+H]; ¹H NMR (400 MHz, CDCl₃) δ (ppm): 9.63 (s, 1H), 7.94-7.72 (m, 4H), 7.48 (d, J=3.1 Hz, 1H), 7.36-7.29 (m, 2H), 6.98 (td, J=8.4, 2.5 Hz, 1H), 6.21 (s, 1H), 4.22-3.92 (m, 5H), 3.68-3.58 (m, 4H), 3.30 (t, J=11.1 Hz, 1H), 2.98-2.92 (m, 2H), 2.58-2.51 (m, 1H), 2.37 (t, J=10.0 Hz, 1H).

### Synthesis of Example 6

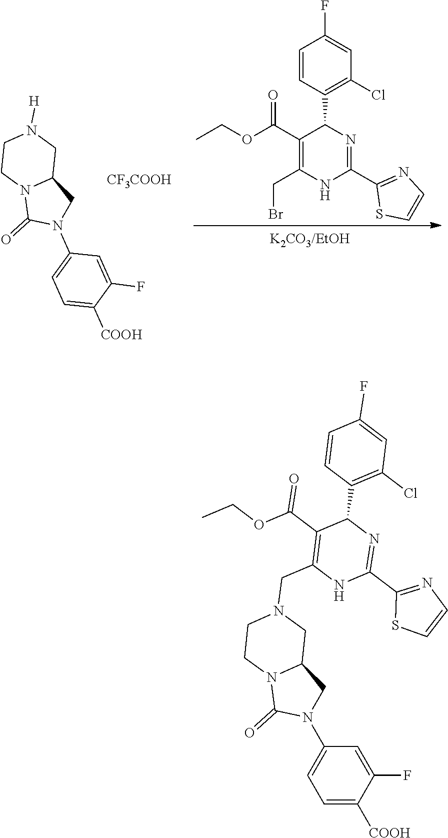 [SMILES: O=C(O)c1ccc(N2C[C@@H]3CNCCN3C2=O)cc1F ; "CF3COOH" ; CCOC(=O)C1=C(CBr)NC(c2nccs2)=N[C@H]1c1ccc(F)cc1Cl ; CCOC(=O)C1=C(CN2CCN3C(=O)N(c4ccc(C(=O)O)c(F)c4)C[C@@H]3C2)NC(c2nccs2)=N[C@H]1c1ccc(F)cc1Cl]

Methyl (R)-6-(Bromomethyl)-4-(2-chloro-4-fluorophenyl)-2-(thiazol-2-yl)-1,4-dihydropyrimidine-5-carboxylate of Example 2 was replaced with ethyl (R)-6-(bromomethyl)-4-(2-chloro-4-fluorophenyl)-2-(thiazol-2-yl)-1,4-dihydropyrimidine-5-carboxylate (0.23 g, 0.5 mmol), and the rest were operated according to the method of Example 2 to obtain a yellow solid (0.13 g, 39.0%). MS (ESI, pos. ion) m/z: 657.1 [M+H]⁺; H NMR (400 MHz, CDCl₃) δ (ppm): 9.57 (s, 1H), 7.98 (t, J=8.5 Hz, 1H), 7.87 (d, J=3.1 Hz, 1H), 7.56 (d, J=13.8 Hz, 1H), 7.48 (d, J=3.0 Hz, 1H), 7.37-7.29 (m, 2H), 7.15 (dd, J=8.5, 2.4 Hz, 1H), 6.94 (td, J=8.4, 2.4 Hz, 1H), 6.24 (s, 1H), 4.17-3.91 (m, 7H), 3.45 (dd, J=9.2, 4.9 Hz, 1H), 3.35-3.25 (m, 1H), 2.98-2.92 (m, 2H), 2.57-2.47 (m, 1H), 2.27 (t, J=10.6 Hz, 1H), 1.14 (t, J=7.1 Hz, 3H).

### Synthesis of Example 7

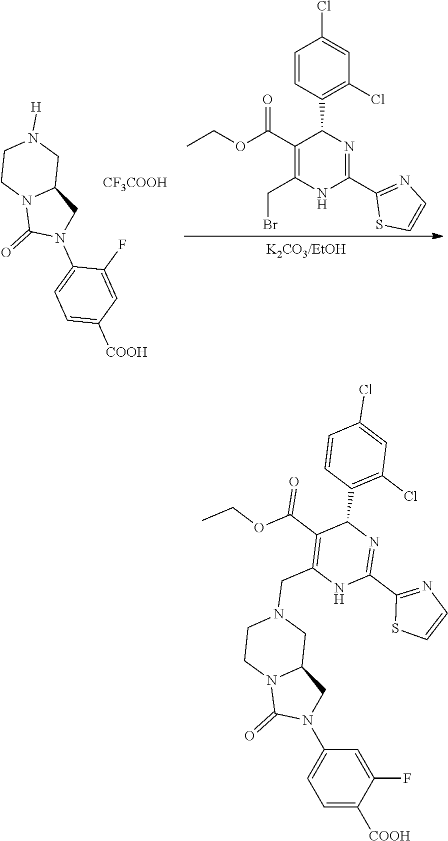 [SMILES: O=C(O)c1ccc(N2C[C@@H]3CNCCN3C2=O)c(F)c1 ; "CF3COOH" ; CCOC(=O)C1=C(CBr)NC(c2nccs2)=N[C@H]1c1ccc(Cl)cc1Cl ; CCOC(=O)C1=C(CN2CCN3C(=O)N(c4ccc(C(=O)O)c(F)c4)C[C@@H]3C2)NC(c2nccs2)=N[C@H]1c1ccc(Cl)cc1Cl]

Methyl (R)-6-(Bromomethyl)-4-(2-chloro-4-fluorophenyl)-2-(thiazol-2-yl)-1,4-dihydropyrimidine-5-carboxylate of Example 1 was replaced with ethyl (R)-6-(bromomethyl)-4-(2,4-dichlorophenyl)-2-(thiazol-2-yl)-1,4-dihydropyrimidine-5-carboxylate (0.38 g, 0.79 mmol), and the rest were operated according to the method of Example 1 to obtain a yellow solid (0.23 g, 43%). MS (ESI, pos. ion) m/z: 673.1 [M+H]; ¹H NMR (400 MHz, CDCl₃) δ (ppm): 7.90-7.75 (m, 4H), 7.47 (d, J=3.1 Hz, 1H), 7.40 (d, J=2.0 Hz, 1H), 7.26 (d, J=1.2 Hz, 1H), 7.19 (dd, J=8.4, 2.0 Hz, 1H), 6.22 (s, 1H), 4.21 (d, J=16.5 Hz, 1H), 4.11-3.92 (m, 6H), 3.64 (dd, J=7.7, 4.4 Hz, 1H), 3.38-3.28 (m, 1H), 3.02-2.94 (m, 2H), 2.58-2.52 (m, 1H), 2.45-2.36 (m, 1H), 1.13 (t, J=7.1 Hz, 3H).

### Synthesis of Example 8

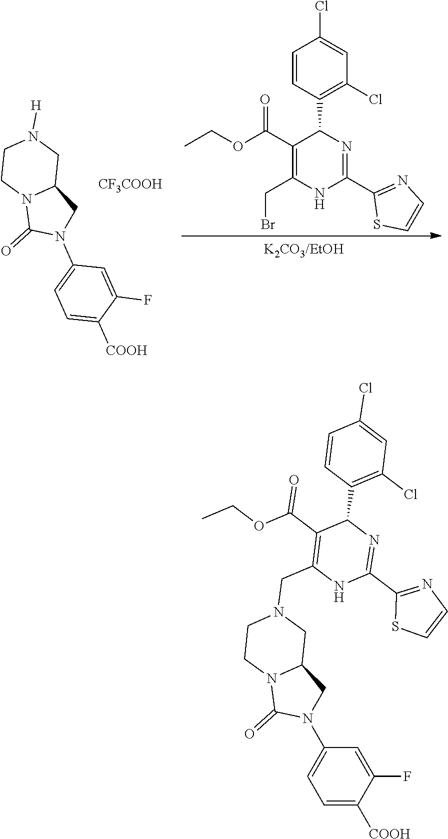 [SMILES: O=C(O)c1ccc(N2C[C@@H]3CNCCN3C2=O)cc1F ; "CF3COOH" ; CCOC(=O)C1=C(CBr)NC(c2nccs2)=N[C@H]1c1ccc(Cl)cc1Cl ; CCOC(=O)C1=C(CN2CCN3C(=O)N(c4ccc(C(=O)O)c(F)c4)C[C@@H]3C2)NC(c2nccs2)=N[C@H]1c1ccc(Cl)cc1Cl]

Methyl (R)-6-(Bromomethyl)-4-(2-chloro-4-fluorophenyl)-2-(thiazol-2-yl)-1,4-dihydropyrimidine-5-carboxylate of Example 2 was replaced with ethyl (R)-6-(bromomethyl)-4-(2,4-dichlorophenyl)-2-(thiazol-2-yl)-1,4-dihydropyrimidine-5-carboxylate (0.38 g, 0.79 mmol), the rest were operated according to the method of Example 2 to obtain a yellow solid (0.26 g, 48%). MS (ESI, pos. ion) m/z: 673.1 [M+H]; H NMR (400 MHz, CD₃OD-d₄) δ (ppm): 7.94 (d, J=3.1 Hz, 1H), 7.89 (t, J=8.7 Hz, 1H), 7.73 (d, J=3.1 Hz, 1H), 7.61 (dd, J=14.1, 1.8 Hz, 1H), 7.46 (d, J=2.0 Hz, 1H), 7.40 (d, J=8.4 Hz, 1H), 7.33-7.26 (m, 2H), 6.17 (s, 1H), 4.15 (d, J=16.9 Hz, 1H), 4.07-3.92 (m, 6H), 3.54 (dd, J=9.4, 4.5 Hz, 1H), 3.30-3.21 (m, 1H), 3.02 (t, J=8.7 Hz, 2H), 2.52-2.42 (m, 1H), 2.24 (t, J=10.8 Hz, 1H), 1.11 (t, J=7.1 Hz, 3H).

Biological Test

Test 1: Evaluation of HepAD38 Cells on the Compound's Inhibitory Activity of HBV DNA Replication (qPCR Method)

HBV Cell Strain and Culture Conditions

HepAD38: Ladner et al. (Ladner, Otto et al. 1997) ligated the tetracycline-sensitive cytomegalovirus CMV promoter to the PBR322 plasmid and ligated it with the ayw subtype HBV DNA into the ptetHBV plasmid, and HepG2 cells were transfected to obtain the HepAD38 cell strain. Due to the destruction of the pre-C region gene, the yield of HBV DNA was about 11 times higher than that of HepG2.2.15 cells. Tetracycline could be used to regulate HBV replication, and the time required for culture was only half of that of HepG2.2.15 cells. It was suitable for studying the HBV replication process and replication intermediates and screening of anti-HBV drugs. HepAD38 was cultured in DMEM/F-12K medium containing 10% FBS and 1% double antibody (it also contained Tetracycline at a final concentration of 300 ng/ml and G418 at a final concentration of 400 μg/ml).

The virus particle DNA secreted by HepAD38 cells could be quantified by qPCR method, and thus the influence of the compound on virus replication could be detected.

Test of Anti HBV Activity In Vitro

After resuscitating the HepAD38 with a small number of passages, after the cells were in good condition, Tetracycline (final concentration of 300 ng/ml) and G418 (final concentration of 400 μg/ml) were added into the medium. The virus did not express in the presence of Tetracycline. After the cells were full, they were digested, counted, and diluted with DMEM/F-12K medium containing 10% FBS (containing Tetracycline at a final concentration of 300 ng/ml and G418 at a final concentration of 400 μg/ml, 1% double antibody) to a cell suspension of concentration of 2×10⁵/mL. The cell suspension was seeded in a 96-well plate (the whole plate was covered) at 100 μL per well, and incubated at 37° C. in a constant temperature incubator with 5% CO₂ for 24 h.

Compound preparation and cell treatment in antiviral experiments: the compound was dissolved with DMSO to 20 mM. Furthermore, the compound was diluted with DMSO to 800 μM, and then 8 dilutions at 4 fold were performed, the highest concentration was 800 μM. 1 μL of serially diluted compound was added to each well of the above-mentioned cell plate. The highest final concentration of the experiment was 4 μM (200 fold dilution). TDF (tenofovir disoproxil fumarate, Selleck, Cat S1400) was used as a positive control compound with a highest concentration of 4 μM. 1 μL of DMSO was added to the negative control wells to a final concentration of 0.5%.

HBV DNA Q-PCR

The one-step hepatitis B virus nucleic acid quantitative determination kit for 48 people (PCR-fluorescent probe method) of Shengxiang Biotechnology was used for QPCR. 2.5 μL of supernatant was absorbed for Q-PCR, and the kit reagents were vortexed and mixed well after the reagents were melted before use. After centrifugation, the enzyme mixture was placed on ice for later use, and it was ensured that the subsequent steps were completed on the ice. 2.5 μL of sample release agent and 2.5 μL of test sample supernatant (experimental group, control group, standard curve group) were added to each well of the Q-PCR plate. After QPCR reaction, the copy number of virus DNA in each well was obtained. Graphpad Prism 5 software was used to process the concentration-virus copy number, and the EC₅₀ of the compound against virus replication was calculated through a four-parameter nonlinear regression model. The experimental results were shown in Table 2.

TABLE 2
EC₅₀ values of the compounds disclosed herein on HBV replication

| Examples | EC₅₀(nM) |
|---|---|
| Example 1 | 20 |
| Example 2 | 10 |
| Example 3 | 31 |
| Example 4 | 16 |
| Example 6 | 15 |
| Example 7 | 16 |
| Example 8 | 13 |

Conclusion: the experimental data showed that the compound disclosed herein had a good inhibitory activity against HBV and had a good application prospect in anti-HBV virus.

Test 2: Evaluation of HepG2.2.15 Cells on the Compound's Inhibitory Activity of HBV DNA Replication

HBV Cell Line and Culture Conditions

The chromosomes of HepG2.2.15 cells (SELLS, PNAS, 1987 and SELLS, JV, 1988) integrated a complete HBV genome and stably expressed viral RNA and viral proteins. HepG2.2.15 cells could secrete mature hepatitis B virus particles, HBsAg and HBeAg into the culture medium. HepG2.2.15 cells were cultured in a DMEM medium containing 10% fetal bovine serum, 100 U/mL penicillin, 100 U/mL streptomycin, 1% non-essential amino acids, 1 mM sodium pyruvate and 300 g/mL G418.

The virus particle DNA secreted by HepG2.2.15 cells could be quantified by qPCR method, and the influence of the compound on virus replication could be detected.

Test of Anti HBV Activity In Vitro

8,000 HepG 2.2.15 cells per well were seeded into a 96-well cell culture plate, the plate was cultured at 37° C. and 5% CO2 for 3 days until the cells grew to full wells. Old liquid medium was discard and replaced with 200 μL of new medium (5% FBS) on day 0.

Compound preparation and cell treatment in antiviral experiments: the compound was dissolved with DMSO to 30 mM. Furthermore, the compound was diluted with DMSO to 800 μM, and then 8 dilutions at 4 fold were performed, the highest concentration was 800 μM. 1 μL of serially diluted compound was added to each well of the above-mentioned cell plate. The highest final concentration of the experiment was 4 μM (200 fold dilution). TDF (tenofovir disoproxil fumarate, Selleck, Cat S1400) was used as a positive control compound with a highest concentration of 4 μM. 1 μL of DMSO was added to the negative control well to a final concentration of 0.5%, and TDF was added to the positive control well to a final concentration of 1 μM.

Detection of Viral Genomic DNA by qPCR

Primers: HBV-For-202, CAGGCGGGGTTTTTCTTGTTGA; HBV-Rev-315, GTGATTGGAGGTTGGGGACTGC. SYBR Premix Ex Taq II—Takara DRRO81S kit was used, and 1 μL cell culture supernatant was used as a template. The plasmid containing the HBV genome was used as a standard curve to calculate the copy number of virus. Graphpad Prism 5 software was used to process the concentration-virus copy number, and the EC₅₀ of the compound against virus replication was calculated through a four-parameter nonlinear regression model. The experimental results were shown in Table 3.

TABLE 3
EC₅₀ values of the compounds disclosed herein on HBV replication

| Examples | EC₅₀(nM) |
|---|---|
| Example 2 | 4 |

Conclusion: the experimental data showed that the compound disclosed herein had a good inhibitory activity against HBV and had a good application prospect in anti-HBV virus.

Test 3: Cytotoxicity and Selectivity Index

Method of testing cytotoxicity and selectivity index of the compound: Serially diluted compound was added to a 384-well cytotoxic cell plate, 50 μL of HepG2.2.15 cells (3000 cells/well) were added to each well, and the maximum final concentration of the experiment was 150 μM (200 fold dilution). The plate was cultured at 37° C. in an incubator with CO2 for 4 days, and cytotoxicity of the compound was detected using CellTiter Glo agent.

The compound cytotoxicity was calculated by the following formula: cytotoxicity (%)=100−(detection value/average value of DMSO control wells×100). The Graphpad Prism 5 software was used to process concentration-cytotoxicity (%) data, and the CC₅₀ was calculated through a four-parameter nonlinear regression model. CC₅₀ greater than 50 indicated relatively low toxicity.

Conclusion: the experimental data of cytotoxicity showed that the compound disclosed herein was less toxic to cells.

Test 4: Pharmacokinetic Experiment of the Compound Disclosed Herein in Beagle Dogs, Mice, Rats, and Cynomolgus Monkeys

(1) PK Test on Beagle Dogs

The PK test method of the compound in vivo of beagle dogs (purchased from Hunan slack Jing Da laboratory animal Co., Ltd., weight 10-12 kg, male, ages of 10-12 months, 3 per oral group, 3 per intravenous injection group): http://www.baidu.com/link?url=7-mSslKxqFR_qMLIOsJKubWFKqZ2430Y-wmsWLpSKHi0P0ubaAo3bIKD2JrsUTqH

The beagle dogs were administered intragastrically with the test compound at doses of 2.5 mg/kg or 5 mg/kg or administered intravenously with the test compound at doses of 1 mg/kg or 2 mg/kg.

Blood samples were taken at 0.083, 0.25, 0.5, 1, 2, 4, 6, 8 and 24 hours from vein after the administration, and collected in anticoagulation tube with EDTA-K2. After liquid-liquid extraction, the plasma sample was quantitatively analyzed on a triple quadrupole tandem mass spectrometer using multiple reactive ion monitoring (MRM). Pharmacokinetic parameters were calculated using a noncompartmental method by WinNonLin 6.3 software.

Conclusion: the pharmacokinetic experiment data showed that the compound disclosed herein had good pharmacokinetic properties in beagle dogs and had a good application prospect in anti-HBV virus.

(2) PK Test on Mice:

The PK test method of the compound in vivo of mice (purchased from Hunan slack Jing Da laboratory animal Co., Ltd., weight: 20-25 g, male, ages of 45-60 days, 3 per oral group, 3 per intravenous injection group):

The ICR mice were administered intragastrically with the test compound at doses of 10 mg/kg or administered intravenously in the tail veins with the test compound at doses of 2 mg/kg or 10 mg/kg.

Blood samples were taken at 0.083, 0.25, 0.5, 1, 2, 4, 6, 8 and 24 hours from orbital vein after the administration, and collected in anticoagulation tube with EDTA-K2. After liquid-liquid extraction, the plasma sample was quantitatively analyzed on a triple quadrupole tandem mass spectrometer using multiple reactive ion monitoring (MRM). Pharmacokinetic parameters were calculated using a noncompartmental method by WinNonLin 6.3 software.

Conclusion: the pharmacokinetic experimental data showed that the compound disclosed herein had good pharmacokinetic properties in mice and had a good application prospect in anti-HBV virus.

(3) PK Test on SD Rats:

The PK test method of the compound in vivo of SD rats (purchased from Hunan slack Jing Da laboratory animal Co., Ltd., weight: 200-250 kg, male, ages of 2-3 months, 3 per oral group, 3 per intravenous injection group).

The Rats were administered intragastrically with the test compound at doses of 2.5 mg/kg or 5 mg/kg or administered intravenously with the test compound at doses of 1 mg/kg.

Blood samples were taken at 0.083, 0.25, 0.5, 1, 2, 5, 7 and 24 hours from vein after the administration, and collected in anticoagulation tube with EDTA-K2. After liquid-liquid extraction, the plasma sample is quantitatively analyzed on a triple quadrupole tandem mass spectrometer using multiple reactive ion monitoring (MRM). Pharmacokinetic parameters were calculated using a noncompartmental method by WinNonLin 6.3 software.

Conclusion: the pharmacokinetic experimental data showed that the compound disclosed herein had good pharmacokinetic properties in SD rats and had a good application prospect in anti-HBV virus.

(4) PK Test on Cynomolgus Monkeys:

The PK test method of the compound in vivo of cynomolgus monkeys (purchased from Guangdong Chunsheng Biotechnology Development Co., Ltd., weight 3-6 kg, male, ages of 4-6 years, 3 per oral group, 3 per intravenous injection group):

The cynomolgus monkeys were administered intragastrically with the test compound at doses of 2.5 mg/kg or 5 mg/kg or administered intravenously with the test compound at doses of 0.5 mg/kg or 1 mg/kg.

Blood samples were taken at 0.083, 0.25, 0.5, 1, 2, 4, 6, 8 and 24 hours from vein after the administration, and collected in anticoagulation tube with EDTA-K2. After liquid-liquid extraction, the plasma sample was quantitatively analyzed on a triple quadrupole tandem mass spectrometer using multiple reactive ion monitoring (MRM). Pharmacokinetic parameters were calculated using a noncompartmental method by WinNonLin 6.3 software. The experimental results were shown in Table 4.

TABLE 4
PK data of compounds in cynomolgus monkeys

| | Route of | Dose | Tₘₐₓ | AUCₗₐₛₜ | F |
|---|---|---|---|---|---|
| Test compound | administration | mg/kg | h | hr*ng/mL | % |
| Control compound | iv | 0.5 | 0.083 | 720 | N/A |
| | po | 2.5 | 1.5 | 647 | 23.6 |
| Example 2 | iv | 0.5 | 0.083 | 1020 | N/A |
| | po | 2.5 | 0.5 | 3380 | 64.8 |

Remarks: for the structure and synthesis method of the control compound, see Example 25 on page 90 of the specification of patent application WO2015132276 (i.e., compound 4-((S)-7-(((R)-6-(2-chloro-4-fluorophenyl)-5-(methoxycarbonyl)-2-(thiazol-2-yl)-3, 6-dihydropyrimidin-4-yl)methyl)-3-oxohexahydroimidazo[1,5-a]pyrazine-2(3H)-yl)benzoic acid).

Conclusion: the pharmacokinetic experimental data showed that the area under the drug-time curve AUClast of the compound herein was larger, and the exposure was better, which indicated that the compound disclosed herein was well absorbed in cynomolgus monkeys and stable in vivo, and had high bioavailability. The pharmacokinetic properties were significantly better than the control compound. Therefore, the compound disclosed herein had good pharmacokinetic properties in cynomolgus monkeys and had a good application prospect in anti-HBV virus.

Test 5: Stability Test of the Compound Disclosed Herein in Liver Microsomes of Different Species

Stability test method of the compound in liver microsome of different species: L of a mixed solution of blank solution and liver microsomes were added to a 96-well plate, and 15 μL of buffer containing the test compound was added to each well. The sample was prepared in duplicate. The plates were preincubated at 37° C. for 10 min, and 15 μL of NADPH solution (8 mM) was added at points in time, the final concentration of the test compound was 1 μM, the concentration of liver microsome was 0.5 mg/mL, the final concentration of NADPH was 2 mM. The plates were incubated for 0, 15, 30, 60 min respectively, after incubation was complete, 150 mL of acetonitrile containing interior label was added to the mixed system. μ The sample diluted with acetonitrile was centrifuged at 4000 rpm for 5 min, and 150 μL of the supernatant was sampled to LC-MS/MS for analysis.

Conclusion: the compound disclosed herein had better stability in liver microsomes of different species.

Test 6: Solubility Test Method

Solubility Test Method of the Compound

Unless otherwise specified, the test sample ground to a fine powder was weighed or the liquid test sample was measured and added into a solvent of certain volume at 25° C.±2° C. The mixture was shaken vigorously for 30 s every other 5 min, and the solubility was observed in 30 min. If there were no visible solute particles or droplets, it was considered as completely dissolved. According to the standards of the Chinese Pharmacopoeia 2015:

Very soluble is that 1 g (mL) of solute can be dissolved completely in a <1 mL of solvent;

Freely soluble is that 1 g (mL) of solute can be dissolved completely in a 1 to <10 mL of solvent;

Soluble is that 1 g (mL) of solute can be dissolved completely in a 10 to <30 mL of solvent;

Sparingly soluble is that 1 g (mL) of solute can be dissolved completely in a 30 to <100 mL of solvent;

Slightly soluble is that 1 g (mL) of solute can be dissolved completely in a 100 to <1000 mL of solvent;

Very slightly soluble is that 1 g (mL) of solute can be dissolved completely in a 1000 to <10000 mL of solvent;

Little or no solubility is that 1 g (mL) of solute can not be dissolved completely in a 10000 mL of solvent.

Conclusion: the experimental data of solubility showed that the compound disclosed herein had better solubility.

Test 7: hERG Test Method

Test Method of the Compound to the Heart

Compound/positive control/negative control, membrane fragment containing hERG channel, and tracer with high affinity to hERG channel were added into a 384-well plate in turn. The plate was incubated at 25° C. and 250 rpm for 4 hours. The fluorescence polarization value of each well was measured by a multifunctional microplate reader. The relative inhibition rate and 50% inhibition concentration (IC50) of the compound on the hERG channel were calculated.

Conclusion: the experimental data of hERG test showed that the compound disclosed herein was less toxic to the heart.

Test 8: Liver Drug Enzyme Induction Test

Cell Culture

All incubations were performed at 37° C. in an incubator with 5% CO2 and 95% humidity.

After resuscitation of cryopreserved human liver cells (Baltimore, Md., USA), cell number and cell viability were measured using trypan blue staining method and cell counter. After counting, the hepatocytes were diluted to 700,000 living cells per milliliter with the preheated seed plate culture medium. The diluted hepatocyte suspension was seeded into a 48-well plate with pre-laying collagen at 0.2 mL/well, and the hepatocyte suspension was incubated in an incubator for at least 4 hours. When the cells were adherent, the seed plate culture medium was replaced with an incubation medium containing 2% base matrigel.

The administration liquid was freshly prepared every day using incubation medium, including the test sample (the concentration was not less than 0.1 μM), positive inducers (omeprazole, phenobarbital, rifampicin) of CYP1A2, CYP2B6 and CYP3A4 obtained through diluting with DMSO stock solution to 1000 fold The administration liquid was listed as following table.

| | | |
|---|---|---|
| | Final concentration | Final concentration |
| Positive inducer | of positive inducer | of organic phase |

| | | | |
|---|---|---|---|
| Omeprazole | 50 | μM | 0.1% DMSO (v/v) |
| Rifampicin | 10 | μM | |
| Phenobarbital | 1000 | μM | |

After the culture system was established, the upper culture medium of the sandwich medium was discarded. 200 μL of the freshly prepared administration liquid (including the test sample, positive control, negative control and matrix control) which had been preheated to 37° C. was added into each cell culture well. The cell culture plate was placed in the incubator and continued to culture for 24 hours. After culturing for 24 hours, the administration liquid was replaced by freshly prepared administration liquid and continued culturing for 24 hours. The entire incubation time was 48 hours. Each drug concentration and control concentration have triplicates.

After the cells were incubated with the administration liquid for 48 hours, the remaining drug solution in the plate was discarded. The cell wells were washed with 0.5 mL of HBSS solution preheated to 37° C. twice. Then to each well was added 100 μL of enzyme labelled substrate liquid preheated to 37° C., the plate was incubated for 30 min. After incubating for 30 minutes, 75 μL of the supernatant sample from each well was added to a 96-well deep well plate containing 150 μL of stop solution. The plate was shaken for 10 minutes and centrifuged at 4° C. and 3220 g for 20 min. Then the supernatant solution was taken and diluted with an aqueous solution containing 0.1% formic acid at a ratio of 1:4. After the diluted sample was shaken for 10 minutes, the amount of metabolite production was detected by liquid chromatography tandem mass spectrometry (LC/MS/MS).

After the enzyme activity detection reaction was over, the remaining solution in the supernatant was discarded, and the cells were washed with 0.5 mL of preheated HBSS. 280 μL of RLT lysis buffer containing 1% β-mercaptoethanol was added to each well, and the plate was sealed and shaken for 10 min. Then the plate was transferred to a −80° C. refrigerator for storage.

Cytotoxicity Test

The potential toxicity of the test sample was evaluated by the release of lactate dehydrogenase (LDH) in liver cells. 100 μL of the administration liquid incubated with liver cells for 24 hours and 48 hours was sampled respectively and the concentration of the lactate dehydrogenase was detected using a commercial LDH kit. The cell lysis solution was used as a positive control, and the incubation medium was used as a blank control.

RNA Analysis and Detection

The sample plate was thawed at room temperature and all samples were transferred to a new 48-well cell culture plate. RNA was extracted by using a fully automatic nucleic acid extraction workstation. The samples more than 10% of total samples were taken out randomly from different position of the sample plate. The OD values of 260 nM and 280 nM were measured by using a ND2000 micro spectrophotometer, and the total RNA purify was determined by calculating the ratio of the two. cDNA was obtained by reverse transcription. Selected genes were quantitatively analysed in real time with CFX Connect™ real-time fluorescent quantitative PCR instrument. The reaction conditions were set as follows: 50° C. for two minutes; 95° C. for ten minutes; 40 cycles of the following two steps: 95° C. for fifteen seconds, 60° C. for one minute. Endogenous control 18S rRNA was as the interior label.

Sample Analysis and Testing

Liquid chromatography tandem mass spectrometry (LC/MS/MS) method was used to determine the concentrations of metabolites of three CYP enzyme substrates (Acetaminophen, Hydroxybupropion and 1′-Hydroxymidazolam) in liver cells after protein precipitation. The analysis method was shown in Table 4.

TABLE 4
LCMS analysis method of induction test

| | |
|---|---|
| Compound name | acetaminophen, hydroxybupropion and 1′-hydroxymidazolam |

Liquid phase method

| Mobile Phase A | Aqueous solution containing 0.1% formic acid | | | |
|---|---|---|---|---|
| Mobile Phase B | Acetonitrile solution containing 0.1% formic acid | | | |
| Column | Acquity UPLC BEH C18 1.7 μm 2.1*50 mm number: 186004044 | | | |
| interior label | acetaminophen-d4 (interior label for acetaminophen), hydroxybupropion-d6 | | | |
| | (interior label for hydroxybupropion) and 1′-hydroxymidazolam-13C3 (interior | | | |
| | label for 1′-hydroxymidazolam) | | | |
| Liquid system | Waters UPLC | | | |
| Autosampler | Waters UPLC | | | |
| Injection volume | 5 μL | | | |
| | | Flow rate | | |
| gradient | Time (min) | (μL/min) | A (%) | B (%) |
| | 0.01 | 600 | 98 | 2 |
| | 0.10 | 600 | 98 | 2 |
| | 1.40 | 600 | 5 | 95 |
| | 1.80 | 600 | 5 | 95 |
| | 1.81 | 600 | 98 | 2 |
| | 2.00 | 600 | 98 | 2 |

Mass spectrometry method

| Mass | API 4000 | | | |
|---|---|---|---|---|
| spectrometry | | | | |
| source of ion | Electrospray ionization | | | |
| Scan mode | Multi-reactive ion monitoring | | | |
| polarity | Positive ions | | | |
| | | Retention | De-clustering | Collision |
| Compound name | Ion pair | Time (min) | voltage (eV) | energy (eV) |
| acetaminophen | 152.1/110.1 | 0.66 | 39 | 23 |
| hydroxybupropion | 256.4/238.0 | 0.80 | 70 | 30 |
| 1′-hydroxymidazolam | 342.1/203.1 | 0.91 | 51 | 35 |
| acetaminophen-d₄ | 156.1/114.2 | 0.65 | 55 | 23 |
| (interior label) | | | | |
| hydroxybupropion-d₆ | 262.3/139.0 | 0.79 | 31 | 30 |
| (interior label) | | | | |
| 1′-hydroxymidazolam- | 347.1/208.1 | 0.91 | 53 | 37 |
| ¹³C₃ (interior label) | | | | |

| | | |
|---|---|---|
| Mass | Collision gas | 20 |
| spectrometry | Air curtain | 55 |
| parameters | Atomizing gas | 60 |
| | Heating auxiliary gas | 5500 |
| | Ion transfer voltage | 600 |
| | Atomization temperature | ON |
| | Heater interface | 10 |
| | Inlet voltage | 10 |
| | Collision chamber outlet | 15 |
| | voltage | |

Gene Expression Data Calculation

This project used the ΔCt relative quantitative method to compare the differences in gene expression between different treatment groups. 18S rRNA as the internal reference gene was used to correct the gene expression of each sample. The Ct value of the target gene minus the Ct value of the internal reference gene was ΔCt, i.e. Ct_{target gene}−Ct₁₈ₛ=ΔCt. ΔCt value of the treatment group subtracted ΔCt value of the blank control group was ΔΔCt, i.e. ΔCt treatment group−ΔCt blank control group=ΔΔCt. Finally, statistical analysis was carried out with the method of 2^{-ΔΔCt} to compare the change of multiples between the treatment group and the blank control group.

Enzyme Activity Data Calculation

The experimental data showed the production of CYP1A2, CYP2B6 and CYP3A4 enzyme metabolites. The change of enzyme activity was shown by comparing the induction multiple of the corresponding cytochrome enzyme in the presence or absence of the compound. The calculation method of the induction multiple and the calculation method of the induction ratio of the control compound were as follows:

Induction multiple=enzyme activity of the sample treated with the test sample/enzyme activity of the sample treated with the matrix control

Induction ratio with the control compound=(induction multiple of the sample treated with the test−1)/(induction multiple of the sample treated with the control compound−1)×100%.

Conclusion: the experimental data of liver drug enzyme induction test showed that the compound disclosed herein basically had no induction effect on liver drug enzyme.

Test 9: Experiment on the Effect of Human Serum on the Anti-HBV Efficacy of the Compound Principle of the Experiment

The chromosomes of HepG2.2.15 cells integrated a complete HBV genome and stably express viral RNA and viral proteins. HepG2.2.15 cells can secrete mature hepatitis B virus particles, HBsAg and HBeAg into the culture medium. The viral DNA secreted by HepG2.2.15 cells can be quantified by qPCR method. Different concentrations of human serum were added while the test compound was being processed. Thereby the effect of human serum on the antiviral efficacy of the compound was detected.

Test Method

Compound Treatment of HepG2.2.15 Cells

Step 1: 15000 HepG2.2.15 cells and 200 μL cell culture medium per well were paved in a 96-well cell culture plate.

Step 2: The plate was incubated at 37° C. in a cell incubator with 5% CO₂ for 3 days until the cells grew to full wells.

Step 3: Old liquid medium was discarded and replaced with 200 μL new medium containing 2% FBS and human serum (HS) with different concentrations (0% HS, 5% HS, 10% HS, 20% HS, 40% HS and 50% HS) on day 0.

Step 4: Compound preparation and cell treatment in antiviral experiments: the compound was dissolved with DMSO to a concentration of 30 mM. Furthermore, the compound solution was diluted with DMSO to a concentration of 800 μM. Then eight dilutions at 4 fold were performed, the highest concentration was 800 μM. 1 μL of the serially diluted compound was added to each well of the cell plate in step 3. The highest final concentration of the experiment was 4 μM (200 fold dilution).

Step 5: The experiment was carried out TDF (tenofovir disoproxil fumarate, Selleck, Cat S1400) as a positive control compound under 2% FBS conditions, with a maximum concentration of 4 μM. 1 μL of DMSO was added to the negative control well, and the final concentration of the experiment was 0.5%.

Step 6: The 96-well cell test plate was incubated at 37° C. in a incubator with CO2 for 11 days. The solution was changed every other day (2, 4, 6, 8, 10 days), and 1 μL of freshly prepared test compound was added, the method was shown in steps 3 to 5.

Step 7: 150 μL of supernatant was sampled from each well at 11th day for qPCR detection of viral DNA.

Step 8: Compound preparation and cell treatment in cytotoxicity experiment: a series of diluted compounds were prepared with Bravo liquid handling system, 11 dilutions at 3 fold were performed, the highest concentration was 30 mM. 0.25 μL of serially diluted compound was added to each well of a 384-well cytotoxic cell plate (Greiner 781098) by using Echo550. HepG2.2.15 cells were prepared and resuspended in a culture medium with different concentrations of human serum (50%, 40%, 20%, 10%, 5%, and 0%). 50 μL (4000 cells) of HepG2.2.15 cells prepared above per well was added to a 384-well cytotoxic cell plate. The highest final concentration of the experiment was 150 μM (200 fold dilution). After 4 days of incubation at 37° C. in an incubator with CO₂, the cytotoxicity test was performed.

Detection of Viral Genomic DNA by qPCR Method

Step 1: The supernatant was diluted 2 folds with DPBS under the experiment condition of 20% HS, the supernatant was diluted 4 folds with DPBS under the experiment condition of 40% HS, the supernatant was diluted 5 folds with DPBS under the experiment condition of 50% HS. After uniformly mixing, 1 μL of sample was taken for qPCR detection.

Step 2: 1 μL of the supernatant under the experimental conditions of 0% HS, 5% HS and 10% HS was sampled directly for qPCR detection.

Step 3: The qPCR reaction system was
prepared as following components:

| | | |
|---|---|---|
| SYBR Premix Ex TaqTM II (2×) | 10 | μL |
| HBV-For-202 (10 μM) | 0.8 | μL |
| HBV-Rev-315 (10 μM) | 0.8 | μL |
| ROX Reference Dye (50×) | 0.4 | μL |
| viral supernatant | 1 | μL |
| the final volume after adding water | 20 | μL |

Step 4: The parameters of ABI ViiA7 qPCR instrument were set as follows

Stage 1:

Reps: 95° C., 30 s, 1 cycle

### Stage 2:

Reps: 95° C., 5 s and 60° C., 34 s, 40 cycles

### Adding the Curve of Dissolution

Detection of Compound Cytotoxicity

Step 1: PromegaCelltiter-Glo reagent was balanced to room temperature.

Step 2: Culture medium in the cytotoxicity experimental plate was discarded, and 50 μL of DPBS was added into each well.

Step 3: 10 μL of CellTiter-Glo reagent was added into each well.

Step 4: The plate was shaken on a vibrator for 2 min.

Step 5: The plate was balanced at room temperature away from light for 10 min.

Step 6: The data was read on the Envision reading board (0.1 sec/well) Analysis of results

The standard curve was plotted based on the plasmids containing the HBV genome (Virus copy number: 2×10E6, 2×10E5, 2×10E4, 2×10E3), and the virus copy number was calculated by the standard curve. Graphpad Prism 5 software was used to process the data and plot a concentration-virus copy number curve, and the EC₅₀ was calculated by a four-parameter nonlinear regression model. Cytotoxicity %=100−(detected value/average value of DMSO control wells×100). The cytotoxicity % data was processed with Graphpad Prism 5 software and the curve was plotted, and the CC₅₀ was calculated by a four-parameter nonlinear regression model.

Conclusion: the experimental data indicated that human serum had little effect on the antiviral efficacy of the compound herein. In the presence of human serum, the compound still had a good HBV inhibitory effect, indicating that the compound can play good antiviral effects in the human body.

Although the general description, specific embodiments and experiments have been used to describe the present invention in detail above, some modifications or improvements can be made on the basis of the present invention, which is obvious to those skilled in the art. Therefore, the modifications and variants all belong to the scopes of the invention without departing from the spirits of the invention.

1.-11. (canceled)

12. A compound having Formula (I) or (Ia), or a stereoisomer, a tautomer, an N-oxide, a solvate, a metabolite, a pharmaceutically acceptable salt or a prodrug thereof, 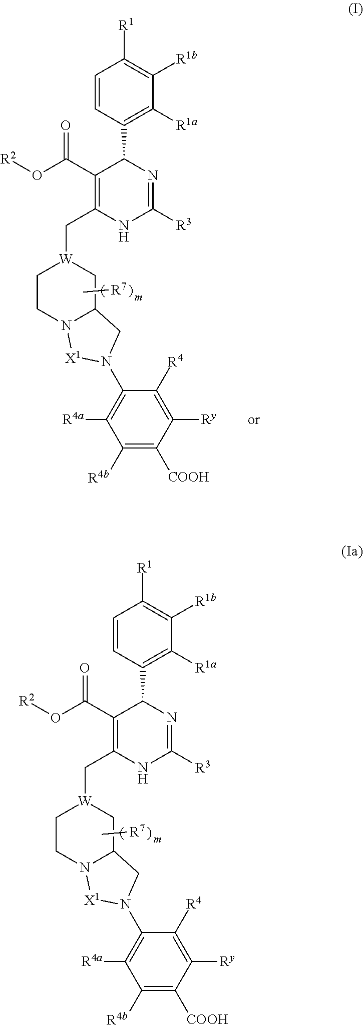
wherein, each of R¹, R^{1b} and R^{1a} is independently hydrogen, deuterium, F, Cl, Br, I, cyano, methyl, ethyl, methoxy, ethoxy, methylamino, ethylamino, nitro, 4-trifluoromethylphenyl, 3,5-bis(trifluoromethyl)phenyl or trifluoromethyl;
R² is C₁₋₆ alkyl or C₁₋₆ haloalkyl;
R³ is phenyl, imidazolyl, furyl, thienyl or thiazolyl, wherein the phenyl, imidazolyl, furyl, thienyl and thiazolyl are each independently unsubstituted or substituted with 1, 2, 3, 4 or 5 substituents selected from the following: deuterium, F, Cl, Br, OH, CN, C₁₋₆ alkyl, hydroxy C₁₋₆ alkyl, C₁₋₆ alkyl-OC(═O)—, C₁₋₆ alkyl-OC(═O)—C₁₋₆ alkylene, HOOC—C₁₋₆ alkylene, C₁₋₆ alkoxy-C₁₋₆ alkylene and C₁₋₆ alkyl-S(═O)₂—;
W is CH or N;
X¹ is —C(═O)—, —S(═O)₂— or —(CR⁵R⁶)ⱼ—;
each of R^{4a}, R^{4b}, R⁵ and R⁶ is independently hydrogen, deuterium, F, Cl, Br, amino, C₁₋₆ alkyl, NH₂C(═O)—, C₁₋₆ alkyl —OC(═O)—, hydroxyl C₁₋₆ alkyl, C₁₋₄ alkoxy C₁₋₄ alkylene or C₁₋₆ haloalkyl;
each R⁷ is independently hydrogen, deuterium, F, Cl, Br, amino, C₁₋₆ alkyl, NH₂C(═O)—, C₁₋₆ alkyl —OC(═O)—, carboxy, carboxy C₁₋₆ alkylene, hydroxy C₁₋₆ alkyl, C₁₋₄ alkoxy C₁₋₄ alkylene or C₁₋₆ haloalkyl;
R¹ is hydrogen, R⁴ is methyl, ethyl, n-propyl, methoxy, ethoxy, n-propoxy, isopropoxy, F or Cl; or
R¹ is F or Cl, R⁴ is hydrogen, F or Cl;
m is 0, 1, 2, 3 or 4;
j is 1, 2, or 3.

13. The compound according to claim 12, wherein R² is methyl, ethyl, n-propyl, isopropyl, monofluoromethyl, difluoromethyl or trifluoromethyl;
R³ is phenyl, imidazolyl, furyl, thienyl or thiazolyl, wherein the phenyl, imidazolyl, furyl, thienyl and thiazolyl are each independently unsubstituted or substituted with 1, 2, 3, 4 or 5 substituents selected from the following: deuterium, F, Cl, Br, OH, CN, methyl, ethyl, n-propyl, isopropyl, tert-butyl, hydroxy C₁₋₄ alkyl, C₁₋₄ alkyl —OC(═O)—, C₁₋₄ alkyl —OC(═O)—C₁₋₃ alkylene, HOOC—C₁₋₃ alkylene, C₁₋₄ alkoxy-C₁₋₃ alkylene and C₁₋₄ alkyl —S(═O)₂—.

14. The compound according to claim 12, wherein each of R^{4a}, R^{4b}, R⁵ and R⁶ is independently hydrogen, deuterium, F, Cl, Br, amino, methyl, ethyl, n-propyl, isopropyl, n-butyl, tert-butyl, NH₂C(═O)—, C₁₋₄ alkyl —OC(═O)—, hydroxy C₁₋₄ alkyl, C₁₋₄ alkoxy C₁₋₂ alkylene or C₁₋₄ haloalkyl;
each R⁷ is independently deuterium, F, Cl, Br, amino, methyl, ethyl, n-propyl, isopropyl, n-butyl, tert-butyl, NH₂C(═O)—, C₁₋₄ alkyl —OC(═O)—, carboxy, carboxy C₁₋₄ alkylene, hydroxy C₁₋₄ alkyl, C₁₋₄ alkoxy C₁₋₂ alkylene or C₁₋₄ haloalkyl.

15. A compound comprising one of the following structures: 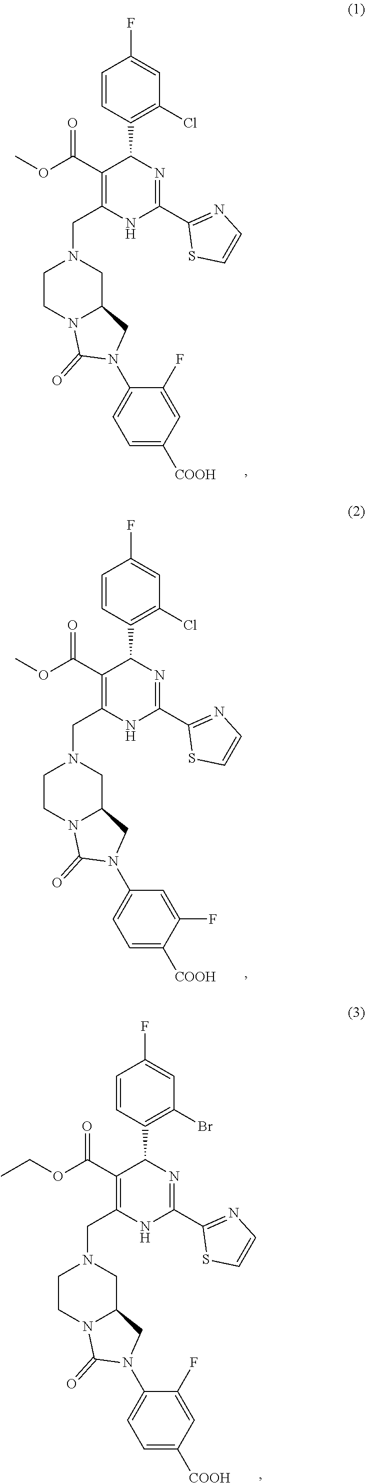 [SMILES: COC(=O)C1=C(CN2CCN3C(=O)N(c4ccc(C(=O)O)cc4F)C[C@@H]3C2)NC(c2nccs2)=N[C@H]1c1ccc(F)cc1Cl ; COC(=O)C1=C(CN2CCN3C(=O)N(c4ccc(C(=O)O)c(F)c4)C[C@@H]3C2)NC(c2nccs2)=N[C@H]1c1ccc(F)cc1Cl ; CCOC(=O)C1=C(CN2CCN3C(=O)N(c4ccc(C(=O)O)cc4F)C[C@@H]3C2)NC(c2nccs2)=N[C@H]1c1ccc(F)cc1Br] 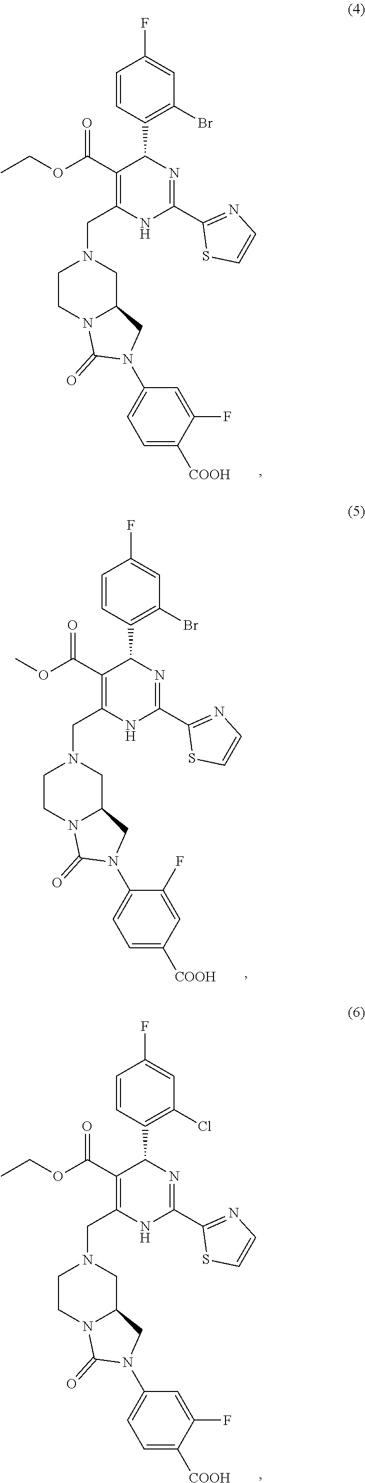 [SMILES: CCOC(=O)C1=C(CN2CCN3C(=O)N(c4ccc(C(=O)O)c(F)c4)C[C@@H]3C2)NC(c2nccs2)=N[C@H]1c1ccc(F)cc1Br ; COC(=O)C1=C(CN2CCN3C(=O)N(c4ccc(C(=O)O)cc4F)C[C@@H]3C2)NC(c2nccs2)=N[C@H]1c1ccc(F)cc1Br ; CCOC(=O)C1=C(CN2CCN3C(=O)N(c4ccc(C(=O)O)c(F)c4)C[C@@H]3C2)NC(c2nccs2)=N[C@H]1c1ccc(F)cc1Cl] 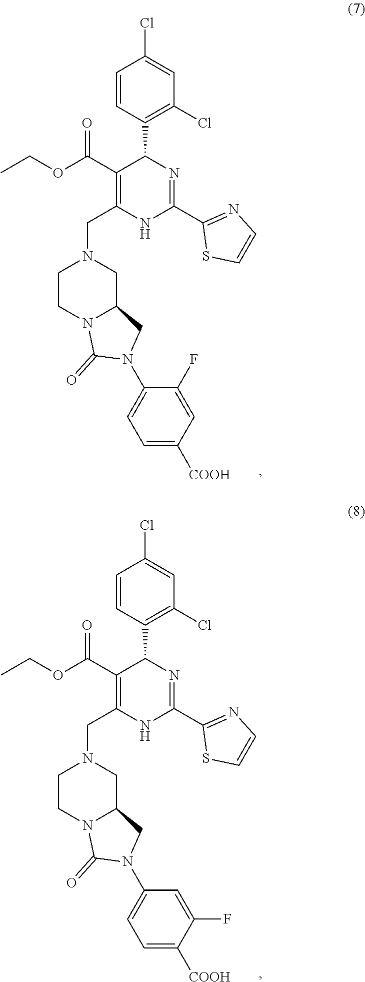 [SMILES: CCOC(=O)C1=C(CN2CCN3C(=O)N(c4ccc(C(=O)O)cc4F)C[C@@H]3C2)NC(c2nccs2)=N[C@H]1c1ccc(Cl)cc1Cl ; CCOC(=O)C1=C(CN2CCN3C(=O)N(c4ccc(C(=O)O)c(F)c4)C[C@@H]3C2)NC(c2nccs2)=N[C@H]1c1ccc(Cl)cc1Cl] 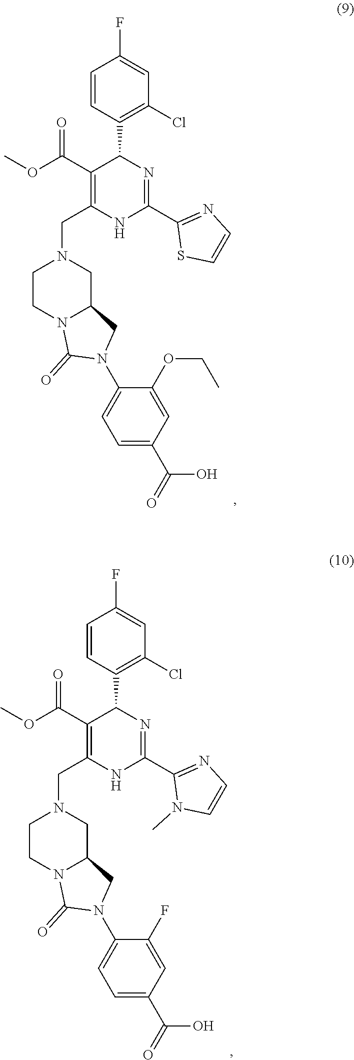 [SMILES: CCOc1cc(C(=O)O)ccc1N1C[C@@H]2CN(CC3=C(C(=O)OC)[C@H](c4ccc(F)cc4Cl)N=C(c4nccs4)N3)CCN2C1=O ; COC(=O)C1=C(CN2CCN3C(=O)N(c4ccc(C(=O)O)cc4F)C[C@@H]3C2)NC(c2nccn2C)=N[C@H]1c1ccc(F)cc1Cl] 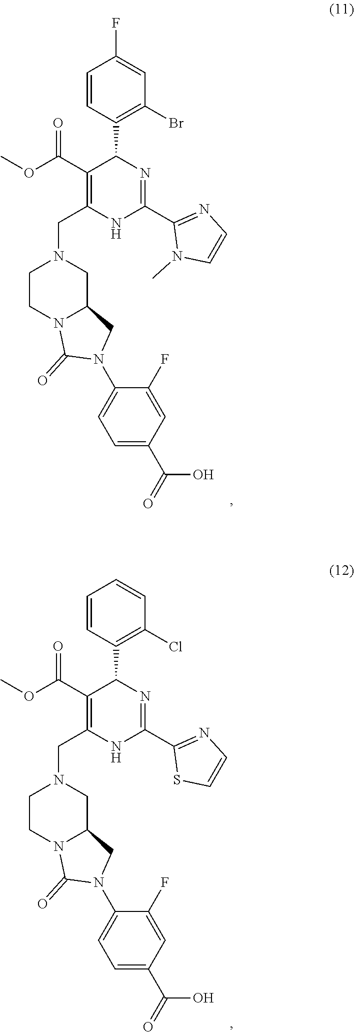 [SMILES: COC(=O)C1=C(CN2CCN3C(=O)N(c4ccc(C(=O)O)cc4F)C[C@@H]3C2)NC(c2nccn2C)=N[C@H]1c1ccc(F)cc1Br ; COC(=O)C1=C(CN2CCN3C(=O)N(c4ccc(C(=O)O)cc4F)C[C@@H]3C2)NC(c2nccs2)=N[C@H]1c1ccccc1Cl] 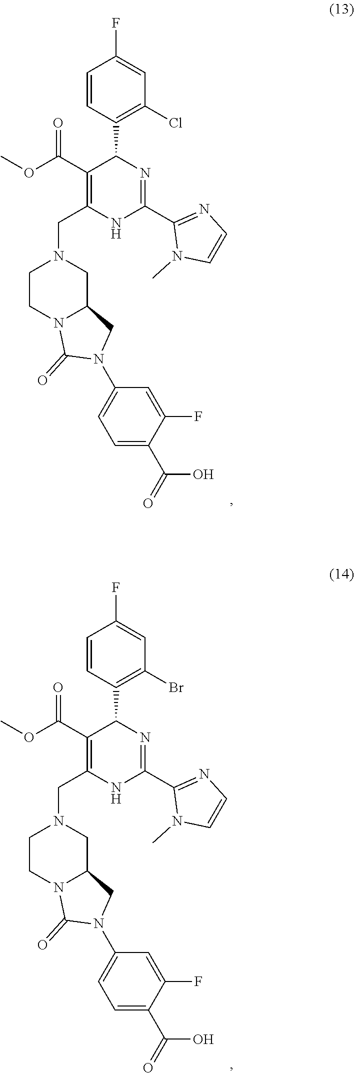 [SMILES: COC(=O)C1=C(CN2CCN3C(=O)N(c4ccc(C(=O)O)c(F)c4)C[C@@H]3C2)NC(c2nccn2C)=N[C@H]1c1ccc(F)cc1Cl ; COC(=O)C1=C(CN2CCN3C(=O)N(c4ccc(C(=O)O)c(F)c4)C[C@@H]3C2)NC(c2nccn2C)=N[C@H]1c1ccc(F)cc1Br] 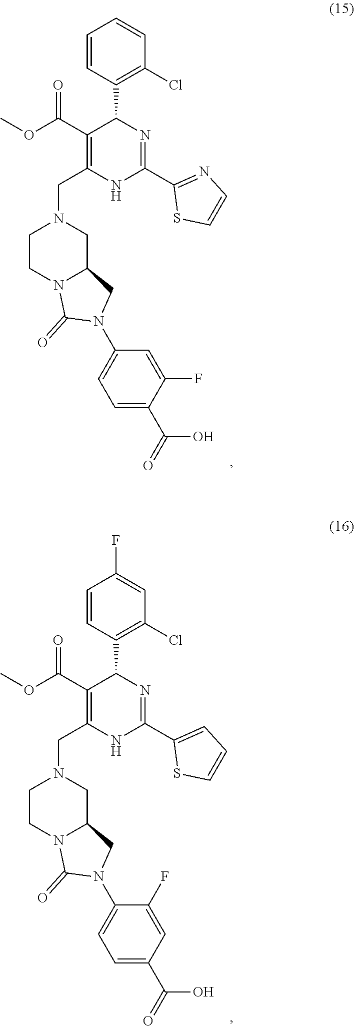 [SMILES: COC(=O)C1=C(CN2CCN3C(=O)N(c4ccc(C(=O)O)c(F)c4)C[C@@H]3C2)NC(c2nccs2)=N[C@H]1c1ccccc1Cl ; COC(=O)C1=C(CN2CCN3C(=O)N(c4ccc(C(=O)O)cc4F)C[C@@H]3C2)NC(c2cccs2)=N[C@H]1c1ccc(F)cc1Cl] 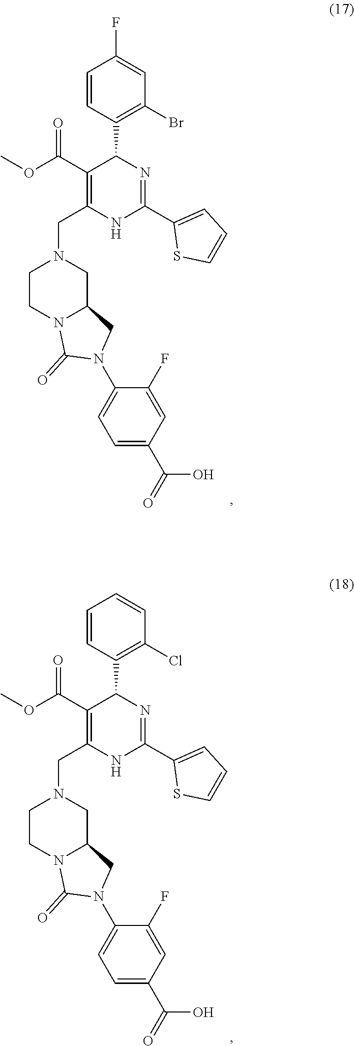 [SMILES: COC(=O)C1=C(CN2CCN3C(=O)N(c4ccc(C(=O)O)cc4F)C[C@@H]3C2)NC(c2cccs2)=N[C@H]1c1ccc(F)cc1Br ; COC(=O)C1=C(CN2CCN3C(=O)N(c4ccc(C(=O)O)cc4F)C[C@@H]3C2)NC(c2cccs2)=N[C@H]1c1ccccc1Cl] 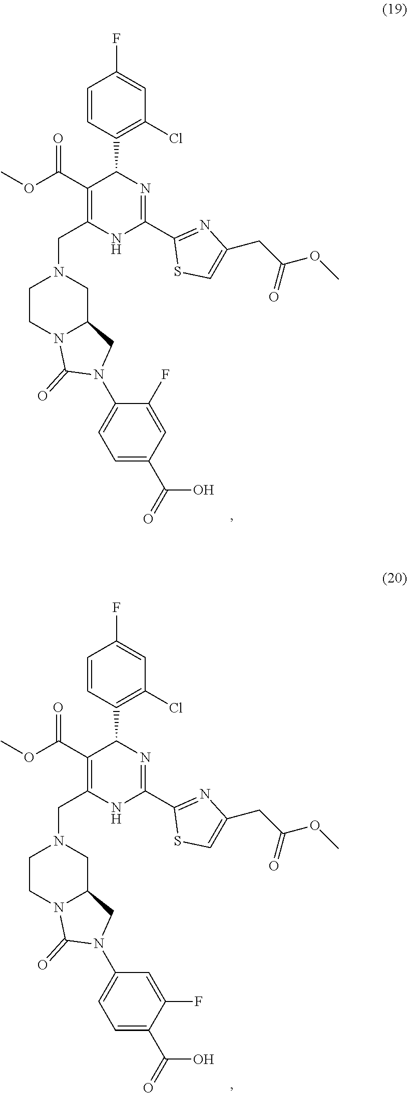 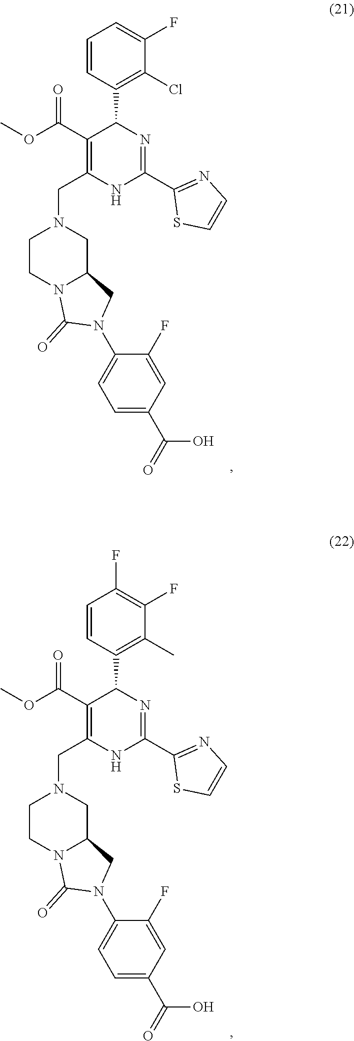 [SMILES: COC(=O)C1=C(CN2CCN3C(=O)N(c4ccc(C(=O)O)cc4F)C[C@@H]3C2)NC(c2nccs2)=N[C@H]1c1cccc(F)c1Cl ; COC(=O)C1=C(CN2CCN3C(=O)N(c4ccc(C(=O)O)cc4F)C[C@@H]3C2)NC(c2nccs2)=N[C@H]1c1ccc(F)c(F)c1C] 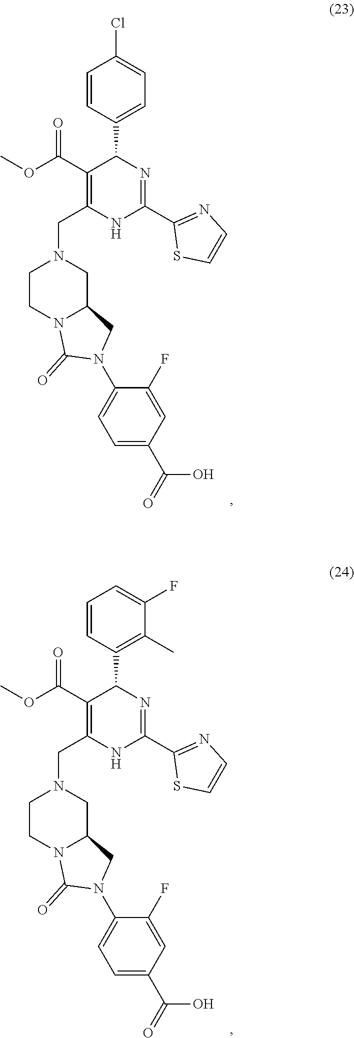 [SMILES: COC(=O)C1=C(CN2CCN3C(=O)N(c4ccc(C(=O)O)cc4F)C[C@@H]3C2)NC(c2nccs2)=N[C@H]1c1ccc(Cl)cc1 ; COC(=O)C1=C(CN2CCN3C(=O)N(c4ccc(C(=O)O)cc4F)C[C@@H]3C2)NC(c2nccs2)=N[C@H]1c1cccc(F)c1C] 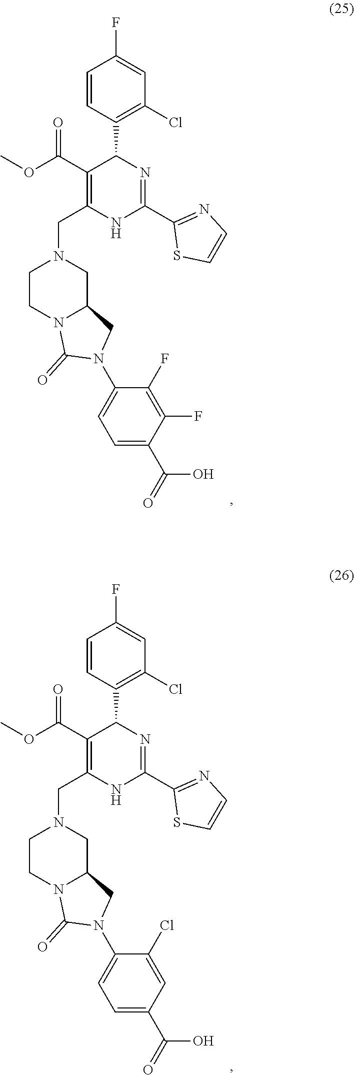 [SMILES: COC(=O)C1=C(CN2CCN3C(=O)N(c4ccc(C(=O)O)c(F)c4F)C[C@@H]3C2)NC(c2nccs2)=N[C@H]1c1ccc(F)cc1Cl ; COC(=O)C1=C(CN2CCN3C(=O)N(c4ccc(C(=O)O)cc4Cl)C[C@@H]3C2)NC(c2nccs2)=N[C@H]1c1ccc(F)cc1Cl] 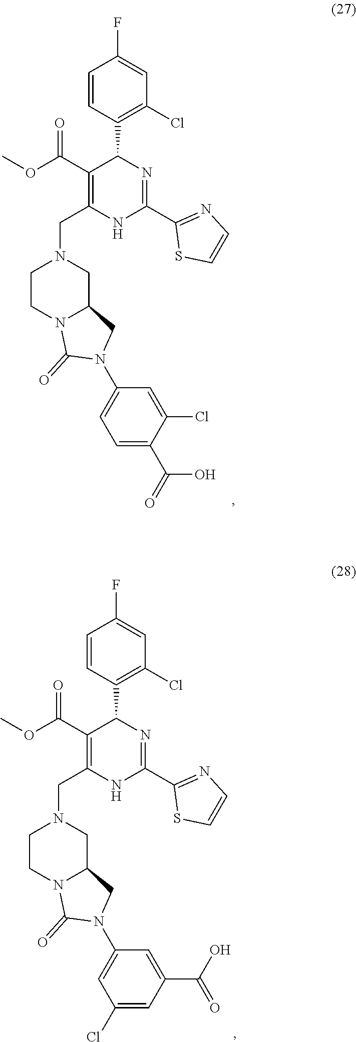 [SMILES: COC(=O)C1=C(CN2CCN3C(=O)N(c4ccc(C(=O)O)c(Cl)c4)C[C@@H]3C2)NC(c2nccs2)=N[C@H]1c1ccc(F)cc1Cl ; COC(=O)C1=C(CN2CCN3C(=O)N(c4cc(Cl)cc(C(=O)O)c4)C[C@@H]3C2)NC(c2nccs2)=N[C@H]1c1ccc(F)cc1Cl] 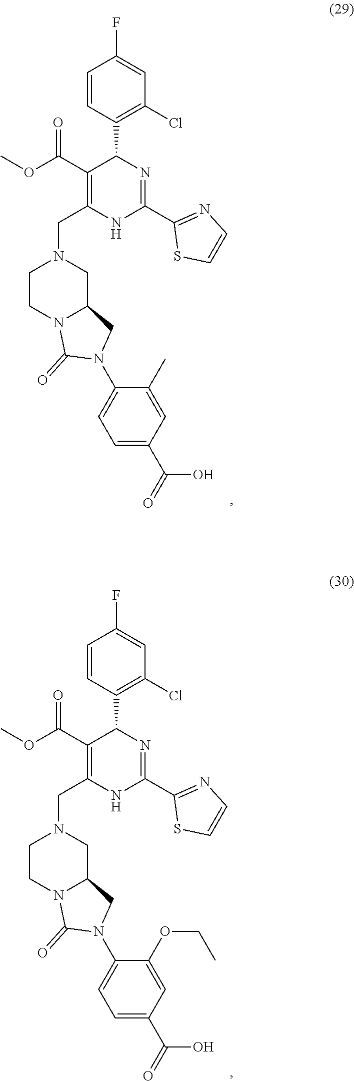 [SMILES: COC(=O)C1=C(CN2CCN3C(=O)N(c4ccc(C(=O)O)cc4C)C[C@@H]3C2)NC(c2nccs2)=N[C@H]1c1ccc(F)cc1Cl ; CCOc1cc(C(=O)O)ccc1N1C[C@@H]2CN(CC3=C(C(=O)OC)[C@H](c4ccc(F)cc4Cl)N=C(c4nccs4)N3)CCN2C1=O] 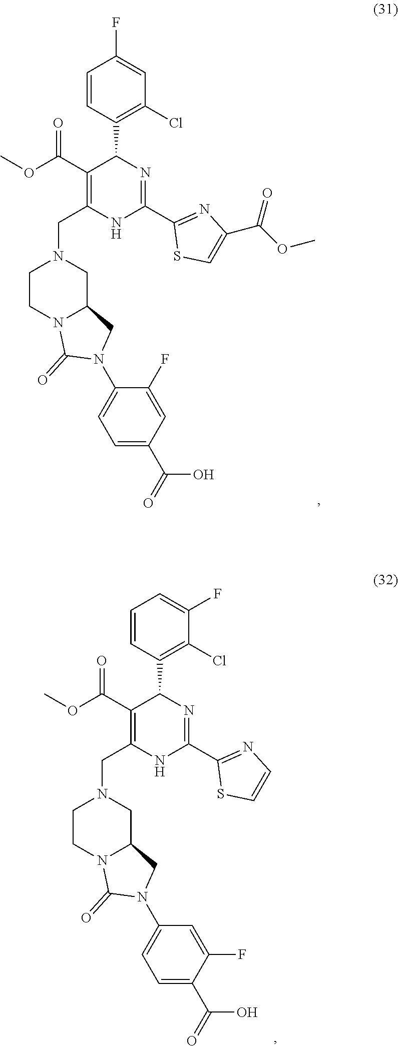 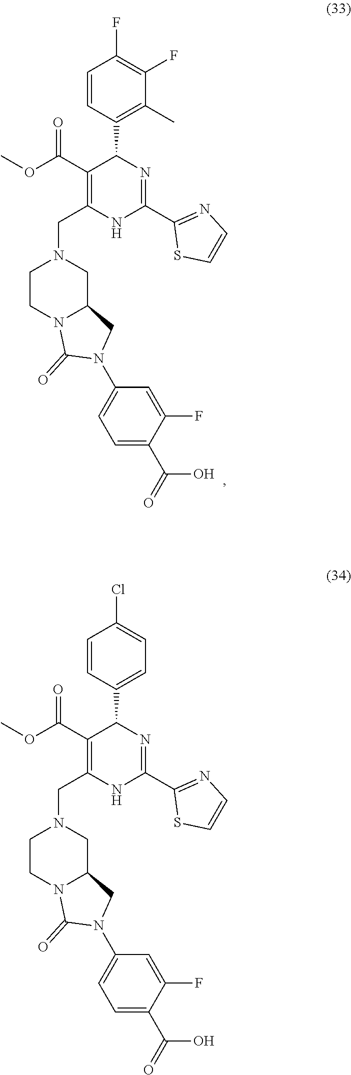 [SMILES: COC(=O)C1=C(CN2CCN3C(=O)N(c4ccc(C(=O)O)c(F)c4)C[C@@H]3C2)NC(c2nccs2)=N[C@H]1c1ccc(F)c(F)c1C ; COC(=O)C1=C(CN2CCN3C(=O)N(c4ccc(C(=O)O)c(F)c4)C[C@@H]3C2)NC(c2nccs2)=N[C@H]1c1ccc(Cl)cc1] 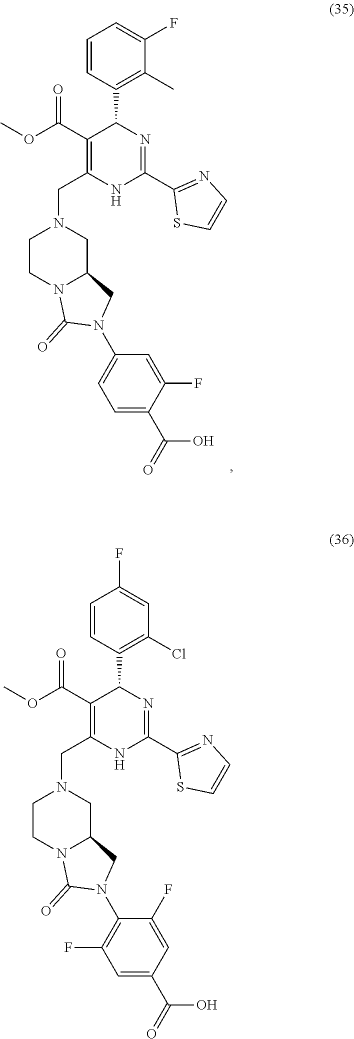 [SMILES: COC(=O)C1=C(CN2CCN3C(=O)N(c4ccc(C(=O)O)c(F)c4)C[C@@H]3C2)NC(c2nccs2)=N[C@H]1c1cccc(F)c1C ; COC(=O)C1=C(CN2CCN3C(=O)N(c4c(F)cc(C(=O)O)cc4F)C[C@@H]3C2)NC(c2nccs2)=N[C@H]1c1ccc(F)cc1Cl]
or a stereoisomer, a tautomer, an N-oxide, a solvate, a metabolite, a pharmaceutically acceptable salt or a prodrug thereof.

16. A pharmaceutical composition comprising the compound of claim 12, and a pharmaceutically acceptable adjuvant.

17. The pharmaceutical composition according to claim 16 further comprising other anti-HBV drug.

18. The pharmaceutical composition according to claim 17, wherein the other anti-HBV drug is a HBV polymerase inhibitor, immunomodulator or interferon, or wherein the other anti-HBV drug is lamivudine, telbivudine, tenofovir, entecavir, adefovir dipivoxil, alfaferone, alloferon, celmoleukin, clevudine, emtricitabine, famciclovir, feron, hepatect CP, intefen, interferon α-1b, interferon α, interferon α-2a, interferon β-1a, interferon α-2, interleukin-2, mivotilate, nitazoxanide, peginterferon α-2a, ribavirin, roferon-A, sizofiran, Euforavac, rintatolimod, Phosphazid, Heplisav, interferon α-2b, levamisole, or propagermanium.

19. A method of preventing, managing, treating or lessening a viral disease in a patient comprising administering to the patient a therapeutically effective amount of the compound according to claim 12.

20. The method according to claim 19, wherein the viral disease is hepatitis B infection or a disease caused by hepatitis B infection, and wherein the disease caused by hepatitis B infection is hepatic cirrhosis or hepatocellular carcinogenesis.

21. A method of preventing, managing, treating or lessening a viral disease in a patient comprising administering to the patient a therapeutically effective amount of the pharmaceutical composition according to claim 16, wherein the viral disease is hepatitis B infection or a disease caused by hepatitis B infection, and wherein the disease caused by hepatitis B infection is hepatic cirrhosis or hepatocellular carcinogenesis.
